(19) United States
(12) Patent Application Publication
(10) Pub. No.: US 2011/0201008 A1
(43) Pub. Date: Aug. 18, 2011

(54) ASSAYS, METHODS AND KITS FOR MEASURING RESPONSE TO THERAPY AND PREDICTING CLINICAL OUTCOME IN PATIENTS WITH B-CELL LYMPHOMA

(75) Inventors: Izidore Lossos, Miami, FL (US); Alvero Alencar, Carlsbad, NM (US); Raquel Malumbres, Huarte (ES); Robert Tibshirani, Stanford, CA (US)

(73) Assignee: UNIVERSITY OF MIAMI, Miami, FL (US)

(21) Appl. No.: 12/957,600

(22) Filed: Dec. 1, 2010

Related U.S. Application Data
(60) Provisional application No. 61/265,567, filed on Dec. 1, 2009.

(51) Int. Cl. C12Q 1/68

(52) U.S. Cl. 435/6.12; 435/6.1; 435/6.17; 435/6.13

(57) ABSTRACT

Assays, methods and kits for predicting survival outcome in a subject having diffuse large B-cell lymphoma (DLBCL) and for measuring a subject's response to DLBCL therapy involve specific miRNAs that are prognostic biomarkers for prediction of outcome of DLBCL patients. Expression of miRNAs miR-18a, miR-181a and miR-222 is associated with response to therapy and outcome of patients with DLBCL. Measurement of these miRs can be used to identify patients' outcomes in the clinic and allow tailoring of patient-specific therapy.

### CROSS-REFERENCE TO RELATED APPLICATIONS

This application claims the benefit of Provisional Application Ser. No. 61/265,567 filed Dec. 1, 2009, which is herein incorporated by reference in its entirety.

### FIELD OF THE INVENTION

The invention relates generally to the fields of cellular biology, oncology, molecular biology, and medicine.

### BACKGROUND

Diffuse large B-ceillymphoma (DLBCL) is a heterogeneous disease with a variable clinical outcome even in the current era of rituximab containing anthracycline-based chemotherapy (RCHOP). Methods of accurate classification of DLBCL and of predicting outcome of patients having DLBCL are greatly needed. Currently, no method for predicting outcome of DLBCL patients is available.

### SUMMARY

Assays, methods and kits for predicting the outcome of a subject (e.g., human) having DLBCL and for measuring a subject's response to DLBCL therapy are described herein. It was discovered that specific miRNAs are prognostic biomarkers for prediction of outcome of DLBCL patients. Predicting the outcome of lymphoma patients is needed for proper therapy selection. Using the methods, kits and assays described herein, a patient's outcome can be predicted within 24 hours of diagnosis of DLBCL. These methods, kits and assays can be used independently, or can be combined with additional diagnostic tests and/or prognostic methods, such as the 6 gene model (Lossos et al. New England Journal of Medicine vol. 350:1829, 2004; Malumbres et al. Blood vol. 111:5509, 2008) and/or the International Prognostic Index (IPI). The assays, methods and kits are robust, and can be used on samples, such as for example, frozen and paraffin embedded specimens (e.g., two scrolls of paraffin with a thickness of 5 microns).

Accordingly, described herein is an assay for predicting survival outcome in a subject (e.g., a human) having diffuse large B-cell lymphoma DLBCL. The assay includes: analyzing expression of miR-18a, miR-181a and miR-222 in a biological sample from the subject; and correlating at least one of: decreased expression of miR-18a in the biological sample compared to a control sample, increased expression of miR-181a in the biological sample compared to a control sample, and decreased expression of miR-222 in the biological sample compared to a control sample with increased survival. A typical method includes correlating decreased expression of miR-18a in the biological sample compared to a control sample, increased expression of miR-181a in the biological sample compared to a control sample, and decreased expression of miR-222 in the biological sample compared to a control sample with increased survival. The biological sample can be, for example, a frozen specimen or a paraffin embedded specimen. Using the assay, the outcome can be predicted within 24 hours of the subject being diagnosed with DLBCL. Expression of miR-18a, miR-181a and miR-222 can be performed, for example, using real-time PCR. The assay can further include analyzing expression of the LMO2, BCL-6, BCL-2, FN-1, CCND-2, and CCL3 genes and calculating a mortality prediction score. The assay can further include determining an International Prognostic Index (IPI) score. In such an assay, the assay includes a multivariate analysis.

In another embodiment, a method for measuring a response to DLBCL therapy (e.g., R-CHOP) in a subject (e.g., a human) having DLBCL is described herein. The method includes: analyzing expression of miR-18a, miR-181a and miR-222 in a biological sample obtained from the subject at a first time point; analyzing expression of miR-18a, miR-181a and miR-222 in a biological sample obtained from the subject at a second time point, the subject receiving the DLBCL therapy after the first time point and before the second time point; comparing the expression of miR-18a, miR-181a and miR-222 in the biological sample obtained from the subject at a first time point to the expression of miR-18a, miR-181a and miR-222 in the biological sample obtained from the subject at a second time point; and correlating at least one of: decreased expression of miR-18a in the biological sample obtained from the subject at a second time point compared to the sample obtained from the subject at a first time point, increased expression of miR-181a in the biological sample obtained from the subject at a second time point compared to the sample obtained from the subject at a first time point, and decreased expression of miR-222 in the biological sample obtained from the subject at a second time point compared to the sample obtained from the subject at a first time point with a therapeutic response to the therapy. The sample obtained from the subject at the first and second time points can be, for example, a frozen specimen or a paraffin embedded specimen.

Also described herein is a kit for predicting survival outcome in a subject having DLBCL. A typical kit includes: at least one reagent (e.g., real-time PCR reagents) for analyzing expression of miR-18a, miR-181a and miR-222 in a biological sample from the subject; at least one control; and instructions for use.

Unless otherwise defined, all technical terms used herein have the same meaning as commonly understood by one of ordinary skill in the art to which this invention belongs.

As used herein, “protein” and “polypeptide” are used synonymously to mean any peptide-linked chain of amino acids, regardless of length or post-translational modification, e.g., glycosylation or phosphorylation.

By the term “gene” is meant a nucleic acid molecule that codes for a particular protein, or in certain cases, a functional or structural RNA molecule.

As used herein, a “nucleic acid” or a “nucleic acid molecule” means a chain of two or more nucleotides such as RNA (ribonucleic acid) and DNA (deoxyribonucleic acid).

The terms “patient,” “subject” and “individual” are used interchangeably herein, and mean a mammalian (e.g., human) subject to be treated and/or to obtain a biological sample from.

As used herein, “bind,” “binds,” or “interacts with” means that one molecule recognizes and adheres to a particular second molecule in a sample or organism, but does not substantially recognize or adhere to other structurally unrelated molecules in the sample. Generally, a first molecule that “specifically binds” a second molecule has a binding affinity greater than about 10⁸ to 10¹² moles/liter for that second molecule and involves precise “hand-in-a-glove” docking interactions that can be covalent and noncovalent (hydrogen bonding, hydrophobic, ionic, and van der waals).

The term “labeled,” with regard to a nucleic acid, protein, probe or antibody, is intended to encompass direct labeling of the nucleic acid, protein, probe or antibody by coupling (i.e., physically or chemically linking) a detectable substance (detectable agent) to the nucleic acid, protein, probe or antibody.

When referring to a nucleic acid molecule or polypeptide, the term “native” refers to a naturally-occurring (e.g., a wild type, WT) nucleic acid or polypeptide.

As used herein, the terms “therapeutic,” and “therapeutic agent” are used interchangeably, and are meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, antibodies, enzymes, peptides organic or inorganic molecules, natural or synthetic compounds and the like.

The term “sample” is used herein in its broadest sense. A sample including polynucleotides, peptides, antibodies and the like may include a bodily fluid, a paraffin embedded specimen, a soluble fraction of a cell preparation or media in which cells were grown, genomic DNA, RNA or cDNA, a cell, a tissue, skin, hair and the like. Examples of samples include saliva, serum, tissue, skin, blood, and plasma. Fresh and frozen samples are described herein.

As used herein, the term “treatment” is defined as the application or administration of a therapeutic agent to a patient or subject, or application or administration of the therapeutic agent to an isolated tissue or cell line from a patient or subject, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

Although assays, kits, and methods similar or equivalent to those described herein can be used in the practice or testing of the present invention, suitable assays, kits, and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a pair of hierarchical clusters showing profiling of miRNA expression in tonsilar subpopulations of lymphocytes by microarray hibridization. (A) Unsupervised hierarchical clustering of tonsilar lymphocyte subpopulations using all miRNAs with no more than 50% missing values. (B) Hierarchical cluster of the same samples performed with the classifier of 39 miRNAs obtained after SAM and PAM analysis. Mean centered log ratios for each miRNA are represented. Missing values are in grey. CB=centroblast, MC=memory B-cell, N=naïve B-cell and T=T-cells.

FIG. 2 is a series of graphs showing paralog clusters miR17/92-1, miR25/106b and miR363/106a are over-expressed in centroblasts. Log ratio values are plotted. Only miRNAs that are the main processed product of the corresponding pre-miRNA and with no more than 3 missing values in the microarrays were included. Dashed lines are used to represent miRNAs not included in the 39 miRNAs cell of origin classifier.

FIG. 3 is a pair of photographs of electrophoretic gels and a series of graphs showing the effects of hsa-miR-223 or hsa-miR-125b over-expression on protein and mRNA levels of genes with roles in B-cell maturation. (A) Effect of the over-expression of hsa-miR-223 on native LMO2 protein levels in VAL cell line at 72 h post transfection, assessed by Western-blot. (B) PRDM1 and IRF4 protein levels detected by Western-blot in RCK8 48 h after transfection of hsa-miR-125b precursor. GAPDH levels were used as loading control in all cases. Data were confirmed in duplicate experiments. Effects of the over-expression of hsa-miR-223 on the mRNA levels of LMO2 (C), or of the over-expression of hsa-miR-125b on mRNA levels of PRDM1 (D left panel up) and IRF4 (D left panel down), measured by Real Time PCR using TaqMan® Gene Expression Assays (Applied Biosystems, Foster City, Calif., USA) at 24, 48 and 72 hours post transfection. Values of triplicate wells are represented as fold expression with respect to the non targeting control transfection. Over-expression of both miRNAs was confirmed by TaqMan® MicroRNA Assays (C right panel and D right panel), expressed as fold increase regarding the control transfection. Error bars correspond to Standard Error of the Mean in all graphs.

FIG. 4 is a series of graphs showing the regulation of expression by hsa-miR-223 or hsa-miR-125b involves binding to specific sites in the 3′-UTRs of LMO2, PRDM1 or IRF4 transcripts. (A) Dual luciferase activity of reporter plasmids with the wild type or mutated 3′-UTR of LMO2 fused to the luciferase gene upon hsa-miR-223 precursor cotransfection in HeLa cells. The same experiment performed for (B) PRDM1 or (C) IRF4 3′-UTR luciferase reporter plasmids after cotransfection with hsa-miR-125b precursor. Black bars represent cotransfections with the corresponding miRNA precursor and white bars the cotransfection of the same reporter vector with the non-targeting control. Values are normalized to the value of each control, that is noted as 100%. Mutation of putative binding sites is expressed as “MUT1” for the most 5′ site, “MUT2” for the most 3′ site and “MUT1+2” for the combined mutation of both sites. Statistical comparisons by two tailed t-test with Bonferroni correction between different constructs are represented as double headed arrows. Statistical comparisons between the cotransfected miRNA and the non-targeting control for the same reporter vector are noted over the black bars. Significant differences with associated P<0.05 are expressed as “*” and non significant difference as “ns”. Error bars correspond to the Standard Error of the Mean.

FIG. 5 is a series of hierarchical clusterings showing miRNA expression profiling of DLBCL cell lines. (A) Unsupervised hierarchical clustering of DLBCL cell line samples using all miRNAs with no more than 50% missing values. (B) Hierarchical clustering of the same samples using only the 9 miRNAs with differential expression between the ABC and GCB DLBCL subtypes based on SAM analysis. (C) The 39 miRNAs cell of origin classifier shown in FIG. 1(B) is used for hierarchical clustering of cell lines and normal tonsilar lymphocyte subpopulations. Mean centered log ratios for each miRNA are represented. Missing values are in grey. CB=centroblast, MC=memory B-cell, N=naïve B-cell, T=T-cells, GCB=Germinal Center-like DLBCL, ABC=Activated B-cell-like DLBCL.

FIG. 6 is a pair of hierarchical clusterings showing cell lines vs. normal centroblasts miRNA expression comparison. (A) Hierarchical clustering of cell lines and their normal counterparts (centroblasts) using the 82 miRNAs with differential expression between these two groups based on SAM analysis. miRNAs comprising the 39 miRNA cell of origin classifier are framed in red; miRNAs with differential expression between DLBCL subtypes are framed in green. (B) Classifier of 5 miRNAs obtained from the PAM algorithm to differentiate DLBCL cell lines from their normal counterparts. Mean centered log ratios for each miRNA are represented. Missing values are in grey. CB=centroblast, GCB=Germinal Center like DLBCL, ABC=Activated B-cell like DLBCL.

FIG. 7 is a pair of graphs showing expression of hsa-miR-222 predicts overall survival (OS) and progression free survival (PFS) in patients with DLBCL treated with R-CHOP chemotherapy. Kaplan-Meier curves for 106 uniformly treated DLBCL patients are plotted. The patients of the cohort were divided into cases with low and high expression based on the median expression of hsa-miR-222. Log-rank test P values are shown.

FIG. 8 is a pair of graphs showing median f/u 2.6 y (0.04-8.1).

FIG. 9 is a pair of graphs showing the IPI score was predictive of both OS and PFS in the cohort of patients.

FIG. 10 is a pair of graphs showing the 6-gene model was also predictive of both OS and PFS in the cohort of patients thus confirming its robustness in an additional cohort of patients.

FIG. 11 is a pair of graphs showing that mir-18a expression is associated with OS as continuous variable.

FIG. 12 is a pair of graphs showing that mir-181a expression is associated with PFS as continuous variable.

FIG. 13 is a pair of graphs showing that mir-222 expression is associated with PFS as categorical variable.

FIG. 14 is a photograph of an electrophoretic gel and a series of graphs pertaining to experiments in VAL cells showing the effects of has-miR-181a expression on protein and mRNA levels of genes with roles in DLBCL-patients' survival.

FIG. 15 is a photograph of an electrophoretic gel and a series of graphs pertaining to experiments in Jukart cells showing the effects of has-miR-181a expression on protein and mRNA levels of genes with roles in DLBCL-patients' survival.

FIG. 16 is a photograph of an electrophoretic gel and a series of graphs pertaining to experiments in Raji cells showing the effects of has-miR-181a expression on protein and mRNA levels of genes with roles in DLBCL-patients' survival.

FIG. 17 is a pair of graphs showing that hsa-miR-181a regulates FOXP1 and CARD11 by binding to cis-regulatory elements in the 3′-UTRs of FOXP1 and CARD11.

### DETAILED DESCRIPTION

Described herein are assays, methods and kits for predicting the outcome of a subject (e.g., human) having DLBCL and for measuring a subject's response to DLBCL therapy. In the experiments described herein, it was demonstrated that miRNAs exhibit specific expressions at different lymphocyte differentiation stages as well as distinct expression in gene expression-defined DLBCL subtypes. The experiments described herein also demonstrate that specific miRNAs are prognostic biomarkers for prediction of outcome of DLBCL patients. In these experiments, RNA was extracted from paraffin-embedded archived specimens (Chen et al Diagn Mol Pathol 2007, 16:61), from patients with newly diagnosed de novo DLBCL treated with RCHOP at 4 different institutions. Expression of 11 miRNAs, including miR-146a, miR-146b-5p, miR-222, miR-500, miR-574-3p, miR-363, miR-155, and miR-21, miR-18a, miR-140-3p, and miR-181a were analyzed by ABI real-time PCR assays. Expression of the genes constituting the 6 gene survival prediction model were measured as reported previously. Expression was correlated to progression-free survival (PFS) and overall survival (OS). The predictive value of miRNAs was evaluated both as a continuous variable as well as a in a dichotomous model with patients grouped based on miRNA median expression. The study group consisted of 176 patients median age 59 years (y) (range 16-92) of which 84 patients (48%) were >60 y. 54 patients (30%) had an ECOG PS ˜2 and 86 (49%) presented with stage III or IV. Distribution according to the IPI was: 0-1 factor (n=77); 2 factors (n=50); 3 factors (n=30); and ˜4 factors (n=19). There were 41 deaths during a median follow-up of 2.6 years (range 0.04-8.1 y). OS correlated with expression of miR-18a as a continuous variable, with higher expression correlating with inferior OS (p=0.038) and was independent of the IPI in a multivariate model. PFS correlated with expression of miR-181a as a continuous variable (p=0.026; higher expression associated with longer PFS) and with expression of miR-222 as a categorical variable (miR-222c; p=0.004, lower expression associated with longer PFS). In a multivariable model analyzing PFS as a variable and including IPI, both miR-222 and miR-181a were IPI independent (p=0.01 and p=0.003, respectively). The mortality-prediction score calculated from the 6-gene model predicted both the OS (p=0.007) and PFS (p=0.004) and was IPI-independent. A multivariate Cox regression analysis that included IPI scores, mortality predictor scores and expression of miR-18a, miR-181a and miR-222c revealed that all factors except miR-222c were independent predictors of OS and all factors except miR-18a were independent predictors of PFS. These results show that expression of miRNAs miR-18a, miR-181a and miR-222 is associated with response to therapy and outcome of patients with DLBCL, independent of the IPI and the mortality-prediction score calculated from the 6-gene model. Measurement of these miRs can be used to identify patients' outcomes in the clinic and allow tailoring of patient-specific therapy.

### Biological and Chemical Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates).

### Assays and Methods of Predicting Survival Outcome in Subjects with DLBCL

Described herein are assays and methods for predicting survival outcome in a subject having DLBCL. Any suitable sample (biological sample) can be analyzed. Examples of samples include paraffin embedded specimens, frozen specimens, biopsy specimens, blood, saliva, serum, plasma, tissue, and urine. A typical method or assay for predicting survival outcome in a subject having DLBCL includes: analyzing expression of miR-18a, miR-181a and miR-222 in a biological sample from the subject; and correlating at least one of: decreased expression of miR-18a in the biological sample compared to a control sample, increased expression of miR-181a in the biological sample compared to a control sample, and decreased expression of miR-222 in the biological sample compared to a control sample with increased survival. In one embodiment, the method or assay includes correlating decreased expression of miR-18a in the biological sample compared to a control sample, increased expression of miR-181a in the biological sample compared to a control sample, and decreased expression of miR-222 in the biological sample compared to a control sample with increased survival. Using the method or assay, a subject's outcome can be predicted within 24 hours of the subject being diagnosed with DLBCL. In the experiments described herein, expression of miR-18a, miR-181a and miR-222 was performed using real-time PCR. However, any suitable methods for analyzing expression of miRs may be used. The method or assay can be a multivariate analysis, and can further include analyzing expression of the genes LMO2, BCL-6, BCL-2, FN-1, CCND-2, and CCL3 and calculating a mortality prediction score (e.g., the 6-gene model as described herein), and can further include determining an International Prognostic Index (IPI) score. Typically, the subject is a mammal such as a human. In some embodiments, at least one subject includes a plurality (e.g., 2, 3, 4, 5, 10, 15, 20, 25, 30, 35, 40, 50, 100, etc.) of subjects who have DLBCL.

### Methods and Assays for Measuring a Response to DLBCL Therapy in a Subject Having DLBCL

The assays and methods described herein can be used for measuring a response to DLBCL therapy in a subject (e.g., human) having DLBCL. One embodiment of a method or assay for measuring a response to DLBCL therapy in a subject having DLBCL includes: analyzing expression of miR-18a, miR-181a and miR-222 in a biological sample obtained from the subject at a first time point; analyzing expression of miR-18a, miR-181a and miR-222 in a biological sample obtained from the subject at a second time point, the subject receiving the DLBCL therapy after the first time point and before the second time point; comparing the expression of miR-18a, miR-181a and miR-222 in the biological sample obtained from the subject at a first time point to the expression of miR-18a, miR-181a and miR-222 in the biological sample obtained from the subject at a second time point; and correlating at least one of: decreased expression of miR-18a in the biological sample obtained from the subject at a second time point compared to the sample obtained from the subject at a first time point, increased expression of miR-181a in the biological sample obtained from the subject at a second time point compared to the sample obtained from the subject at a first time point, and decreased expression of miR-222 in the biological sample obtained from the subject at a second time point compared to the sample obtained from the subject at a first time point with a therapeutic response to the therapy. In addition to the first and second time points, expression of miR-18a, miR-181a and miR-222 can be analyzed in biological samples taken from the subject at other time points, e.g., third, fourth, fifth, sixth time points, etc. For example, if multiple courses of therapy are administered to the subject, expression of miR-18a, miR-181a and miR-222 can be analyzed in biological samples taken from the subject after each course of therapy. To measure a response to a particular therapy, a measurement(s) may be taken at any suitable time (e.g., at the time of diagnosis). One example of a DLBCL therapy is R-CHOP. The sample obtained from the subject at the first and second time points can be any suitable sample, such as a frozen specimen or a paraffin embedded specimen.

### Kits

Described herein are kits for predicting survival outcome in a subject having DLBCL. A typical kit includes: at least one reagent (e.g., real-time PCR reagents) for analyzing expression of miR-18a, miR-181a and miR-222 in a biological sample from the subject; at least one control; and instructions for use. Such kits can also be used, for example, to measure a subject's response to DLBCL therapy. Optionally, kits may also contain one or more of the following: containers which include positive controls, containers which include negative controls, photographs or images of representative examples of positive results and photographs or images of representative examples of negative results.

### Data and Analysis

Use of the assays, methods and kits described herein may employ conventional biology methods, software and systems. Useful computer software products typically include computer readable medium having computer-executable instructions for performing logic steps of a method. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

The assays, methods and kits described herein may also make use of various computer program products and software for a variety of purposes, such as reagent design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170. Additionally, the embodiments described herein include methods for providing data (e.g., experimental results, analyses) and other types of information over networks such as the Internet.

### EXAMPLES

The present invention is further illustrated by the following specific examples. The examples are provided for illustration only and should not be construed as limiting the scope of the invention in any way.

### Example 1

### Differentiation-Stage-Specific Expression of MicroRNAs in B-Lymphocytes and Diffuse Large B-Cell Lymphomas

miRNAs represent a novel class of small functional, noncoding RNAs of 21-23 nucleotides. miRNAs initiate inhibition of translation or degradation of mRNAs by binding to partially complementary sites in the 3′ untranslated region (UTR) of the target mRNA and are emerging as key players in the posttranscriptional regulation of intracellular protein concentrations. miRNAs orchestrate various cellular functions and play critical roles in many biological processes, including cell differentiation, apoptosis, proliferation and cancer development. Expression profiling studies have detected specific miRNA expression “signatures” in a variety of human cancers, and miRNA coding sequences are frequently located at genomic regions associated with cell transformation and carcinogenesis.

Expression of certain miRNAs is differentiation/maturation stage specific, as demonstrated in several compartments of the mammalian hematopoietic precursors and during T-cell differentiation. Peripheral B-cell development and differentiation following immune stimulation are complex processes controlled by distinct programs of transcriptional control. In response to antigen encounter, uncommitted naïve B-cells are activated and undergo a complex maturational process yielding phenotypically distinct subpopulations which form highly organized germinal centers (GC) in lymphoid organs. Within the GC, B-cells undergo high rate proliferation and affinity maturation, are selected by antigen, switch toward mature isotypes and finally differentiate into either memory or plasma cells. This maturation process is characterized by tightly regulated suppression or increased expression of specific genes, resulting in distinctive gene expression signatures at specific differentiation stages (Klein et al., Proc Natl Acad Sci USA. 2003; 100:2639-2644). It is possible that spatio-temporal regulation of miRNA expression also occurs in B lymphocyte lineage during the immune response. A systematic understanding of the roles of miRNAs in this process is incomplete, as few direct studies of changes of miRNA expression over the course of peripheral B-cell differentiation have been conducted. Dynamic regulation of distinct miRNAs within specific B-cell ontogeny stages might influence the maturation process while deregulations of this process might result in block of differentiation and/or malignant transformation.

Genome-wide expression profiling was performed with miRNA arrays in purified normal peripheral B-cell subpopulations (centroblasts, naïve and memory B-cells) along with T-cells. Distinct changes in the expression of specific miRNAs and paralog families at various stages of B-cell development were demonstrated. A specific differential expression ‘signature” of miRNAs was observed in GC lymphocytes (centroblasts) while the differences between naïve (pre-GC) and memory B-cells (post-GC) were less remarkable, similar to previous mRNA expression patterns in these B-cell developmental stages. Enrichment or depletion of specific miRNAs in GC cells can be correlated with corresponding reduction or increase in expression of proteins which mRNA transcripts harbor seed matches to these miRNAs. These findings suggest potential functional importance of temporal regulation of miRNA expression during B-cell differentiation. Indeed, miRNA transfection experiments and 3′-UTR reporter assays demonstrated that GC-enriched hsa-miR-125b down-regulates the expression of IRF4 and PRDM1/BLIMP1—key transcription factors whose expression is repressed in centroblasts but is necessary for differentiation into memory and plasma cells (Klein et al., Nature Immunol. 7:773-782, 2006; Angelin-Duclos et al., J. Immunol. 165:5462-5471, 2000; Kuo et al., J. Exp Med. 204:819-830, 2007). In contrast, GC-depleted but memory B-cell enriched hsa-miR-223 down-regulates the expression of LMO2—a transcriptional factor specifically expressed in GC lymphocytes (Natkunam et al., Blood. 109:1636-1642, 2007). Microarrays were used to characterize miRNA expression profiles in GC B-cell like (GCB) and Activated B-cell (ABC)-like DLBCL. These studies confirmed that although an important component of the biology of a malignant cell is inherited from its non-transformed cellular progenitor—GC centroblasts, aberrant miRNA expression is acquired upon cell transformation and may play an important role in lymphomagenesis. Interestingly, while GCB-like and ABC-like DLBCL differ in expression of hundreds of mRNAs, a 9 miRNA signature was identified that could perfectly differentiate the two major subtypes of DLBCL.

### Material and Methods

### Cell Subpopulations and Cell Lines

Lymphocyte subpopulations were enriched from human reactive tonsils as described in supplemental methods below. Five ABC-like DLBCL cell lines (RIVA, Oci-Ly3, Oci-Ly10, HBL1 and U2932) and three GCB-like DLBCL cell lines (Oci-Ly7, Oci-Ly19 and SUDHL-6) were cultured in IMDM (Cellgro, Herndon, Va.) with 20% human plasma, 1% penicillin/streptomycin/L-glutamine (Cellgro, Herndon, Va.) and 0.2% beta mercaptoethanol (Invitrogen Gibco BRL, Grand Island, N.Y.). HeLa cells were cultured in Dulbecco's Modified Eagle Medium with high glucose (Invitrogen Gibco BRL, Grand Island, N.Y.) and 10% fetal bovine serum (Hyclone, Logan, Utah) and 1% penicillin/streptomycin/L-glutamine (Cellgro, Herndon, Va.).

RNA Isolation and miRNA Microarray Profiling

Total RNA was extracted using the mirVana™ miRNA Isolation Kit (Ambion, Austin, Tex.). Dual colour hybridization microarray experiments for miRNA expression were performed using LC Sciences array platform containing probes for all human miRNAs present in miRBase (Griffiths-Jones et al., Nucleic Acids Res. 34:D140-144, 2006) at the time of the experiment, as previously reported (Wang et al., PLoS ONE. 3:e2557, 2008). Design and analysis of microarray experiments are detailed in supplemental methods below. The data from both microarray experiments is available at GEO database, with accession numbers GSE12934 and GSE12933. To confirm the array miRNA expression results, expression of selected miRNAs was assessed by TaqMan® MicroRNA Assays and the 7900 HT Fast Real-Time PCR System (Applied Biosystems, Foster City, Calif., USA). The expression of RNU6B was used as internal control and 2^{-ΔCt} values were used for expression analysis.

Search for miRNA Target Genes and Analysis of miRNA Clusters

Three prediction algorithms: PicTar (Krek et al., Nat. Genet. 37:495-500, 2005), miRanda (Enright et al., PLoS Biol. 2:e363, 2004) and TargetScan (Lewis et al., Cell. 115:787-798, 2003; Grimson et al., Mol Cell 27:91-105, 2007; Lewis et al. Cell 120:15-20, 2005), were used to find possible targets of the miRNAs involved in the GC reaction among proteins playing an important role in GC reaction and B-cell maturation (e.g. IRF4, PRDM1, BCL2, LMO2). In addition, the PITA algorithm (Kertesz et al., Nat. Genet. 39:1278-1284, 2007) (see parameters in supplemental methods below) was used to confirm the accessibility of putative miRNA binding sites.

miRNA clusters were defined using the miRGen software (Megraw et al. Nucleic Acids Res. 35:D149-155, 2007) as groups of miRNAs located in the same genomic region and not separated by a known gene or more than 5000 bp.

### Transfection Assays and Western Blots

For luciferase reporter experiments, HeLa cells were transfected in triplicate with each 3′ UTR-luciferase construct using SiPort NeoFX (Ambion, Austin, Tex.) according to the manufacturer's instructions as summarized in supplemental methods below. Each experiment was repeated at least 4 times (N=12). Statistical comparison of luciferase results was performed by two tailed t-test with Bonferroni correction and α=0.05. VAL and RCK-8 DLBCL cell lines were transfected by Nucleofector II (Amaxa Biosystems) following manufacturer's instructions as described is supplemental methods below. Cells were collected 48 and 72 hours post-transfection and protein levels were measured by Western-blot. Whole cell lysates of transfected VAL and RCK8 cells were blotted for LMO2 using a monoclonal antibody and for IRF4 and PRDM1 using the IRF4 (P713) antibody (Cell Signaling Technologies, Beverly, Mass.) and BLIMP1/PRDM1 antibody (ABCAM, Cambridge, Mass.), respectively.

Correlation of miRNA Expression and Patient Outcome

A total of 106 specimens from DLBCL patients treated at the British Columbia Cancer Agency (79 patients) and University of Miami (27 patients) were studied. Total RNA was extracted from two 5-μm thick slices of formalin-fixed, paraffin-embedded sections as previously reported (Chen et al., Diagn Mol Pathol. 16:61-72, 2007). Overall survival (OS) was defined as the time interval between the date of diagnoses to the date of death or last follow-up. Progression-free survival (PFS) was defined as the time interval between the date of initial diagnosis and the date of disease progression, death from any cause, or last follow-up evaluation. OS and PFS of patients were estimated using the Kaplan-Meier product-limit method and compared by the log-rank test. P values of less than 0.05 were considered statistically significant.

### Supplemental Methods

Purification of lymphocyte subpopulations from human tonsils: each of the GC centroblasts, naïve and memory B-cell lymphocyte subpopulations were purified from 4 human tonsils as described (Klein et al. Proc Natl Acad Sci USA. 100:2639-2644, 2003) with minor modification—use of the AutoMACS system (Miltenyi Biotec, Auburn, Calif.). T cells were also enriched from 3 tonsils using anti-CD3 conjugated with FITC (Beckman-Coulter, Fullerton, Calif.) and anti-FITC magnetic beads followed by POSSELD positive selection on the AutoMACS system, obtaining more than 90% purity as determined by flow cytometry analysis. Patient samples were obtained from routine tonsillectomies after informed consent and tissue collection was approved by the institutional review board.

Microarray experimental design and analysis: FirstChoice® Human Skeletal Muscle Total RNA (Ambion, Austin, Tex.) was used as the common reference for all the experiments and was labeled with Cy5, while lymphocyte samples were labeled with Cy3. B-cell subpopulations and T cells miRNA profiling was performed with arrays version miRHuman_{—}9.1_{—}070207 containing 470 miRNAs, while cell line profiling was done with version miRHuman_{—}10.0_{—}070802 containing 711 miRNAs. The probes for hsa-miR-377 and hsa-miR-542-5p were excluded from the analysis according to manufacturer's recommendations.

The median normalized expression ratios were log² transformed and the probes with less than 50% missing values across the samples were used for statistical comparison. The remaining missing values were imputed using 5-nearest neighbor imputation prior to assessment of significance using the Significance Analysis of Microarrays (SAM) package samr v1.24 in the R language v2.62 (Tusher et al., Proc Natl Acad Sci USA. 98:5116-5121, 2001). Classification of samples was carried out using Prediction Analysis of Microarrays (PAM) pamr v1.25 in the R language (Tibshirani et al., Proc Natl Acad Sci USA. 99:6567-6572, 2002). Default values were used for all tuning parameters.

Hierarchical clustering of samples and probes was performed with Cluster v2.11 software and visualized with Treeview v1.60 (Eisen et al., Proc Natl Acad Sci USA. 95:14863-14868, 1998). Log ratio values for each miRNA were mean centered before performing average linkage with uncentered correlation coefficient.

PITA algorithm parameters: for the PITA algorithm, a seed of 8 nucleotides was selected, allowing a mismatch and a wobble pair. In the case of hsa-miR-223 binding sites accessibility prediction for LMO2, the number of nucleotides in the seed parameter was reduced to 7 because the only site obtained with seed of 8 nucleotides had a ddG close to 0. For further analysis, putative binding sites were selected with a negative increment of free energy upon miRNA binding (ddG <0).

DNA Constructs: the 3′-UTR regions of LMO2 and IRF4 genes were amplified from T cell genomic DNA and the 3′-UTR of PRDM1 was amplified from memory B-cells cDNA using the Phusion™ High-Fidelity PCR Master Mix (Finnzymes Oy, Espoo, Finland) according to the manufacturer's instructions. PCR products were digested with either NheI-PRDM1- or XbaI-LMO2 and IRF4- (New England Biolabs, Beverly, Mass.) and ligated into the pGL3 Control vector (Promega, Madison, Wis., Madison, Wis.) linearized with XbaI and dephosphorilated with Shrimp Alkaline Phosphatase (USB, Cleveland, Ohio). Ligation products were grown in Top10 F′ E. coli (Invitrogen Gibco BRL, Grand Island, N.Y.) and individual clones were picked up. The sequence of the clones was confirmed using the Taq DyeDideoxy terminator method with the 3130x1 Genetic Analyzer DNA Sequencer (Applied Biosystems, Foster City, Calif., USA).

Mutagenesis of the 3′-UTR luciferase constructs was performed using the QuickChange® Site-Directed Mutagenesis Kit (Stratagene, La Jolla, Calif.) for LMO2 and the QuickChange® XL Site-Directed Mutagenesis Kit (Stratagene, La Jolla, Calif.) for IRF4 and one of the mutated sites of PRDM1. The 3′ putative binding site for hsa-miR-125b in the PRDM1 3′-UTR was located 5′ to a polyA sequence and was mutated using a previously described strategy based on PCR (Cormack B. Directed mutagenesis using the polymerase chain reaction: introduction of point mutations by sequential PCR steps. In: F. M. Ausubel R B, R. E. Kingston, D. D. Moore, J. C. Seidman, J. A. Smith and K. Struhl, ed. Current Protocols in Molecular Biology. New York: Wiley; 1997:8.5.7-8.5.10). Amplifications were performed with Phusion™ High-Fidelity PCR Master Mix (Finnzymes Oy, Espoo, Finland). The obtained fragment was digested with HpaI and XbaI (New England Biolabs, Beverly, Mass.) and subcloned into the luciferase construct containing the 3′-UTR of PRDM1 digested with the same endonucleases and treated with Antarctic Phosphatase (New England Biolabs, Beverly, Mass.).

SiPort transfections: 45,000 cells per well were seeded over 50 μl of transfection mix, in a final volume of 0.5 ml. The transfection mix was prepared using 1 μl of SiPort, 0.4 μg of luciferase pGL3 control derived construct, 80 ng of pRL-TK (Promega, Madison, Wis.) and 5 pmol of pre-miR (Ambion, Austin, Tex.) per well. Cells were lysed 16-24 hours after transfection and Dual Luciferase Assay (Promega, Madison, Wis.) was performed in a Sirius luminometer (Berthold, Aliquippa, Pa.). Measured luciferase expression values were normalized to non-targeting pre-miR negative control #1 (Ambion, Austin, Tex.) which measurement was given value of 100%. Data are presented as average±standard error of the mean.

Nucleofector transfections: 2.5 million cells were transfected with 2 μg of the corresponding pre-miR-223, pre-miR-125b or pre-miR negative control #1 (Ambion, Austin, Tex.). For VAL cells, the program X-001 and solution V were used while for RCK-8 cell the program was H-024 and the solution was L.

Clinical samples selection criteria: the specimens were selected based on the following criteria: (1) diagnosis of de novo DLBCL clinical stages I-IV; (2) availability of tissue obtained at diagnosis before initiation of therapy; (3) treatment with curative intent with rituximab, cyclophosphamide, doxorubicin, vincristine and prednisone (R-CHOP); and (4) availability of follow-up and outcome data at the treating institution. Institutional Review Board approval was obtained from all participating institutions for inclusion of anonymized data in this study. The following information at the time of diagnosis was collected: age, gender, performance status, stage, number of extranodal sites involved, serum lactate dehydrogenase (LDH) level, the presence or absence of systemic (“B”) symptoms, and IPI score. Staging was done in all the patients according to the Ann Arbor system (Carbone et al., Cancer Res. 31:1860-1861, 1971) based on physical examination, bone marrow biopsy, and computed tomography of the chest, abdomen, and pelvis. Follow-up information was obtained from the patients' medical records.

### Results

miRNA Expression in B-Cell Subpopulations

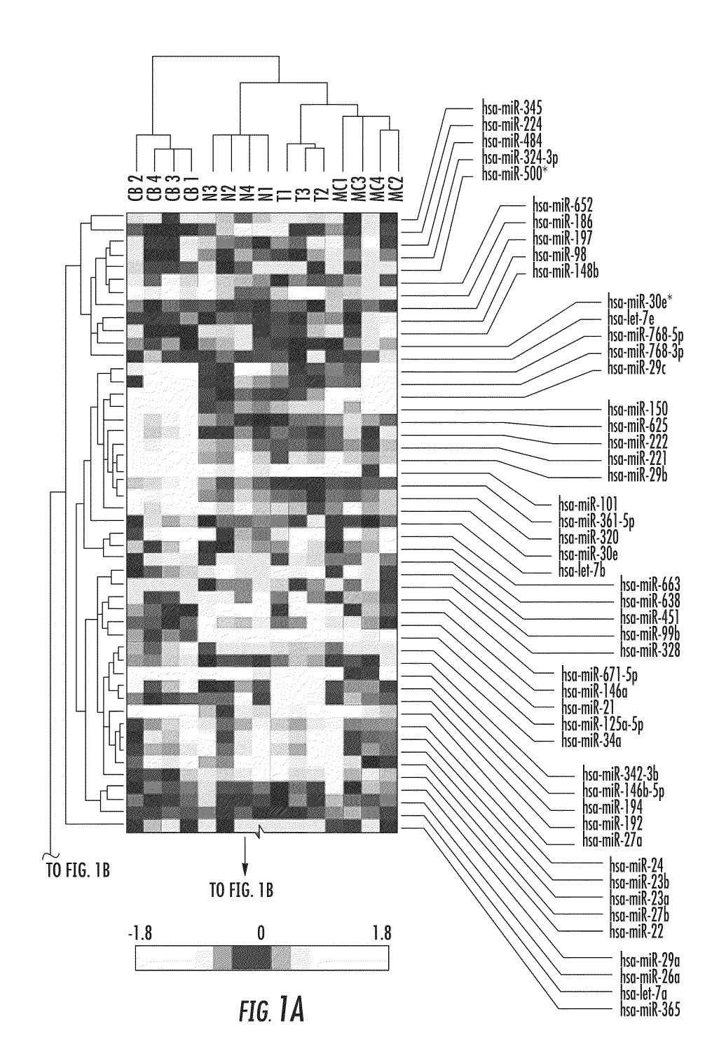

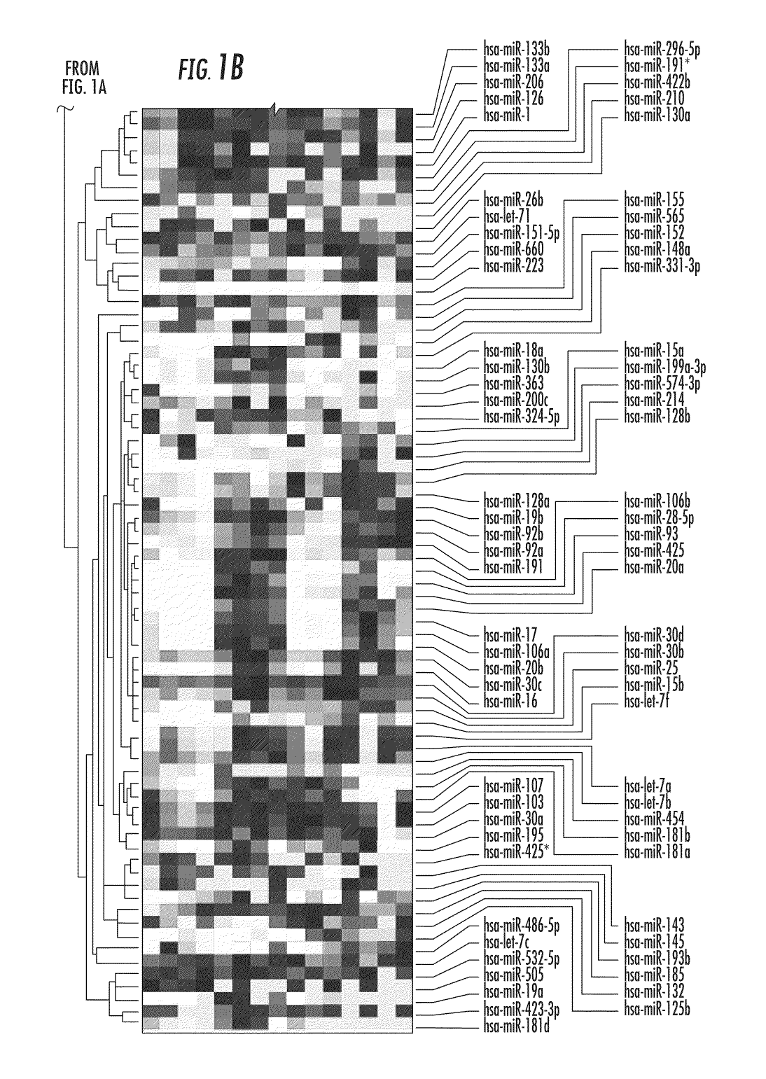

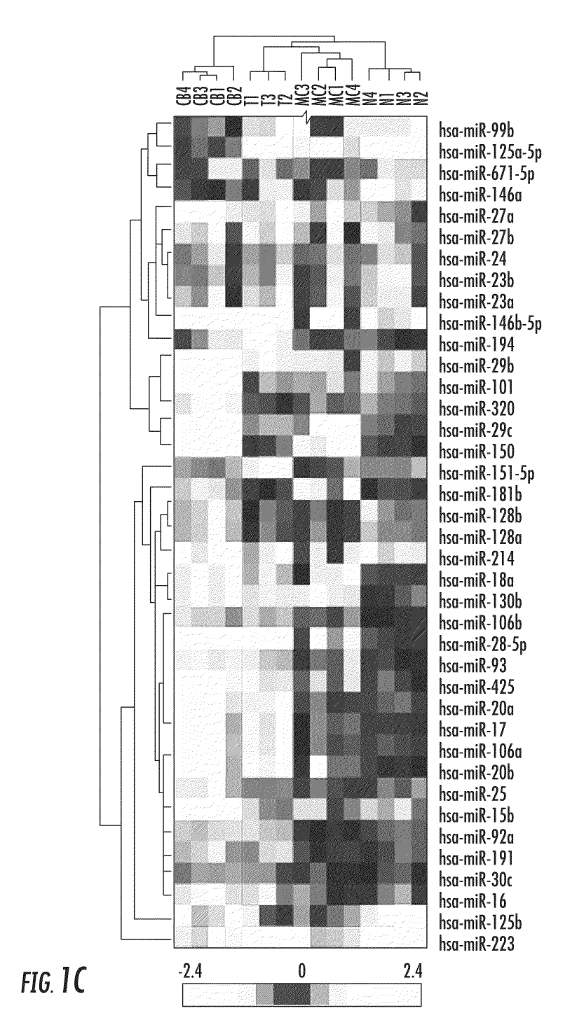

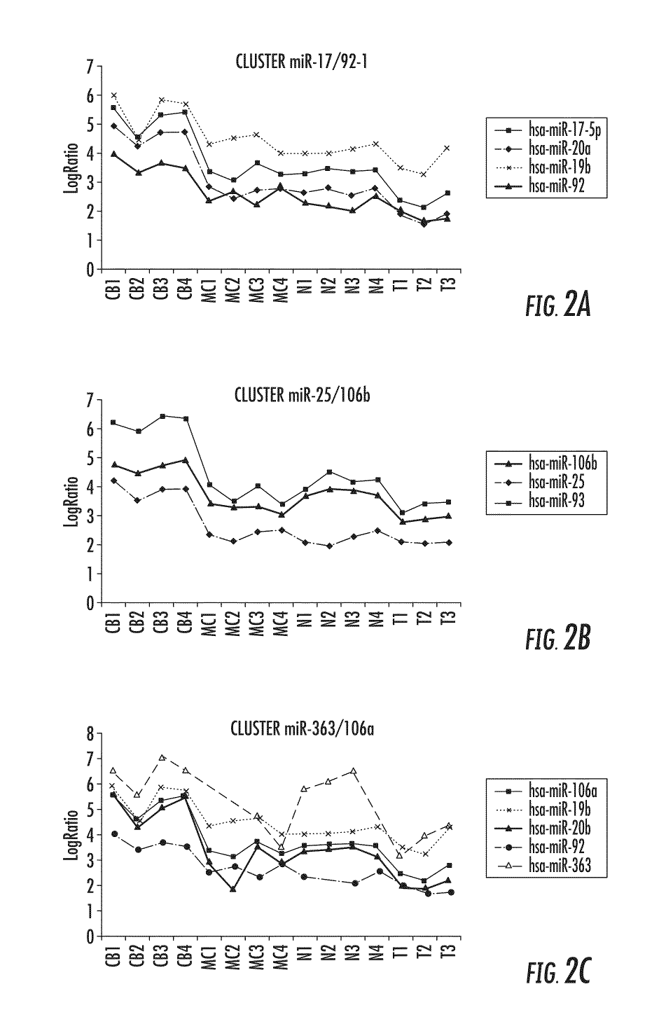

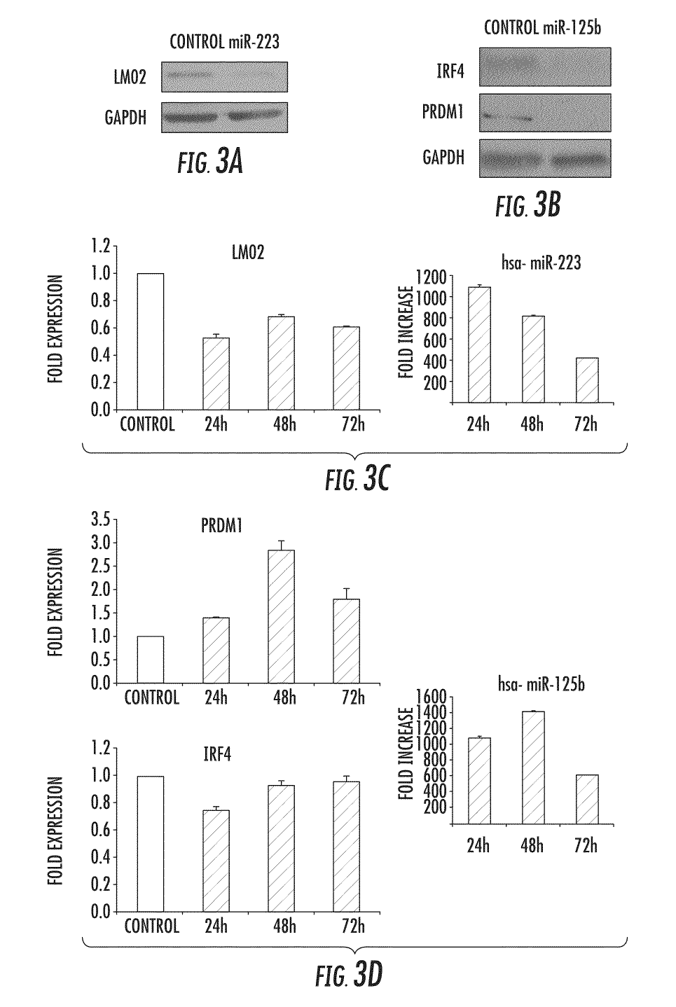

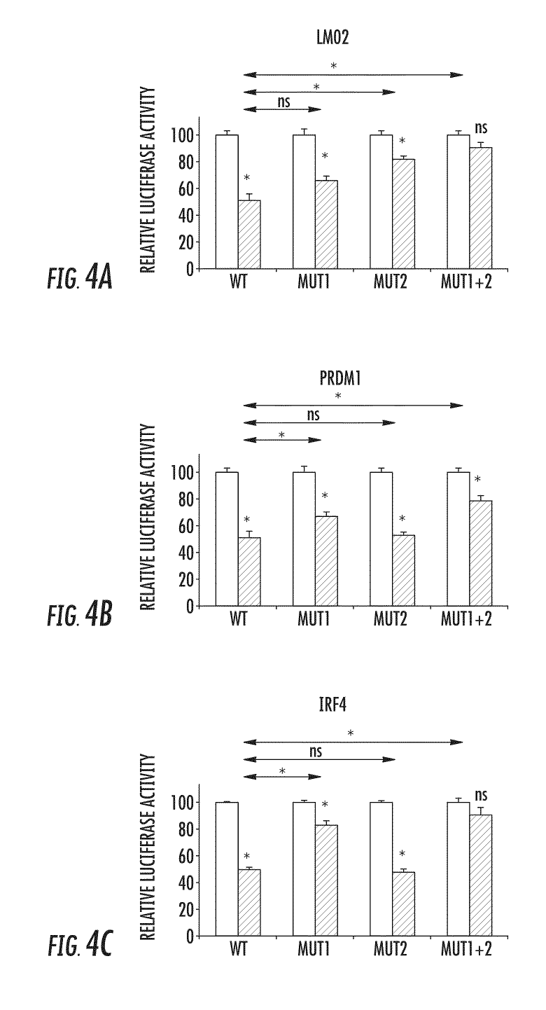

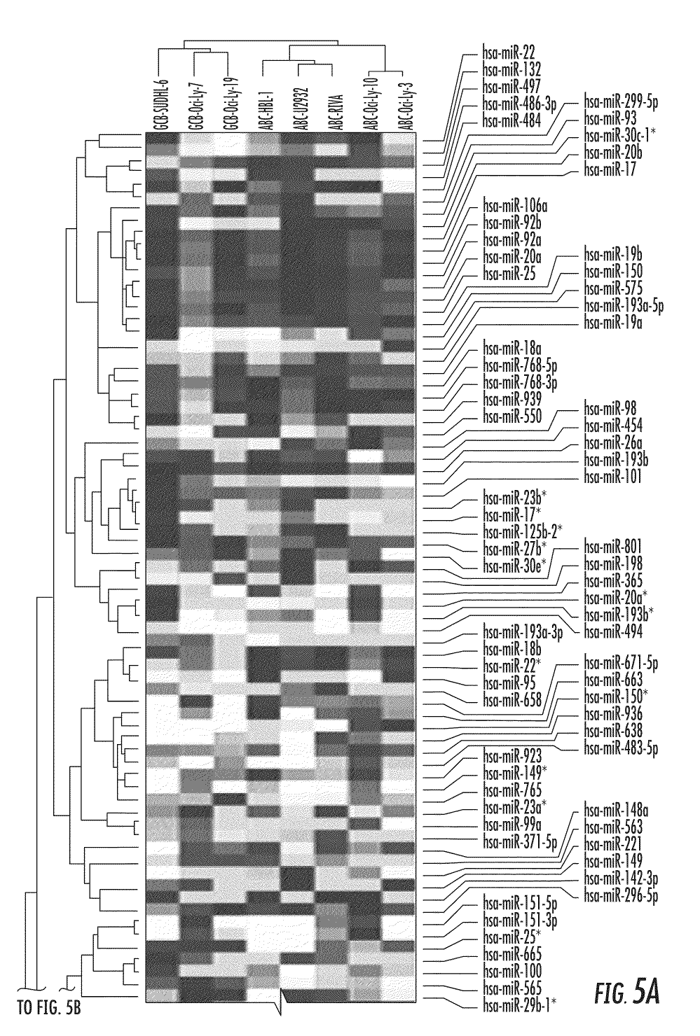

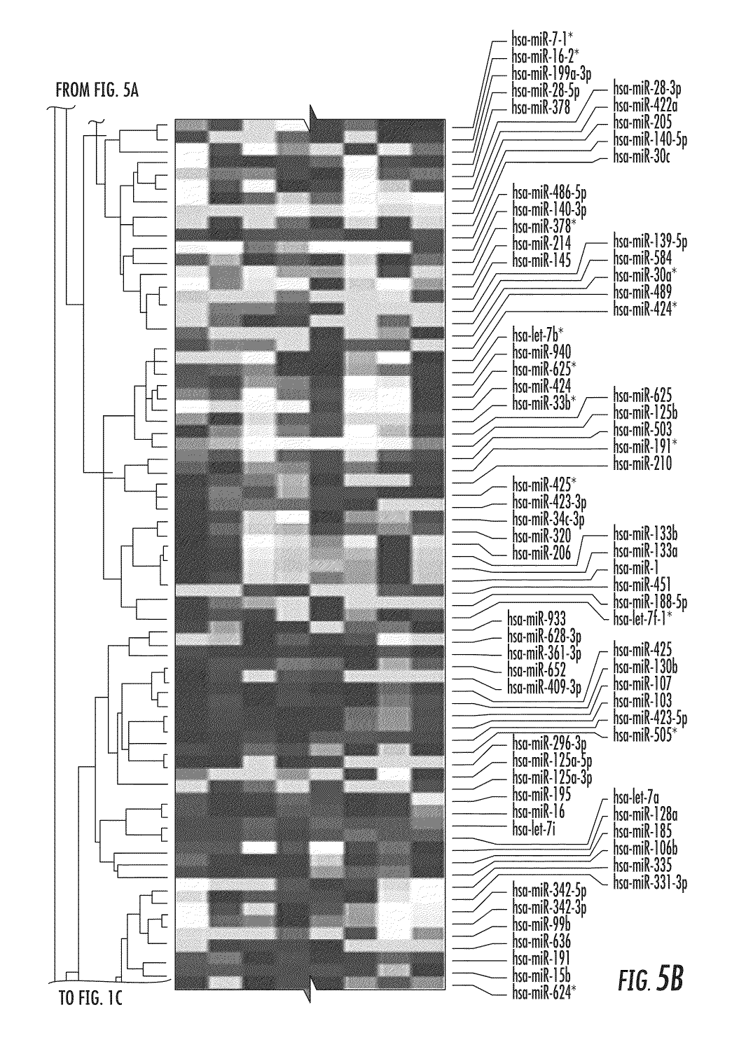

miRNA samples from the purified naive, centroblasts and memory B-cells were labelled and hybridized to LC Sciences arrays representing the 470 miRNAs identified at the time of the experiment. To provide a control for comparison, miRNA expression in T-cells purified from tonsils was also profiled. The 15 data sets corresponding to four samples each of the naïve, centroblasts and memory B-cells and three samples of T-cells were analyzed by unsupervised hierarchical clustering. This algorithm perfectly segregated B-cell subpopulations and T-cells based on global similarities in miRNA expression patterns (FIG. 1A). Remarkably, the pattern of miRNA expression in centroblasts was clearly distinguishable from the other cell subpopulations, with specific increased expression of 51 miRNAs. Although naïve and memory B-cells could be distinguished based on their miRNA expression pattern, the observed differences were less striking compared to the GC lymphocytes. T-cell miRNA expression pattern could be distinguished from patterns observed in B-cell subpopulations; however, it showed closest resemblance to the miRNA expression pattern detected in memory B-cells. Taken together, these findings demonstrate that each of the purified B-cell subpopulations displays a distinct miRNA expression profile that is consistent among different individuals.

To identify a miRNA classifier sufficient to separate individual B-cell subpopulations from each other and also from T-cells, a Significance Analysis of Microarrays (SAM) (Proc Natl Acad Sci USA. 98:5116-5121, 2001) was performed using the miRNA probes with data in at least 50% of samples. This approach yielded 118 differentially expressed miRNAs with a false discovery rate (FDR) of 10%. Next, a Prediction Analysis of Microarrays (PAM) (Tibshirani et al., Proc Natl Acad Sci USA. 99:6567-6572, 2002) was applied and this yielded a cell of origin classifier based on 39 miRNAs with one miss-classification error, as estimated by cross-validation. Hierarchical clustering of all the 15 cell subpopulation samples using these miRNAs resulted in heat map correctly separating the four lymphocyte subpopulations (FIG. 1B). Twenty one of the 39 miRNAs in this cell of origin classifier are specifically up-regulated in centroblasts. Notably, tremendous changes in miRNA expression upon B-cell maturation were observed for specific miRNAs. For example, the expressions of hsa-miR-18a and hsa-miR-28 were 15 and 10 fold higher, respectively, in centroblasts compared to memory B-cells, while an opposite trend of markedly higher expression (10 to 20 fold) in memory B-cells compared to centroblasts was observed for hsa-miR-101c, hsa-miR-150 and hsa-miR-29c. To confirm the LC array expression data, 9 miRNAs were selected from the classifier and their expression was measured in cell subpopulation samples using TaqMan MicroRNA Assays. An excellent correlation with array data was observed.

Some miRNAs are organized in clusters that are transcribed as a single pri-miRNA. miRNA members of the same cluster frequently exhibit similar patterns of expression. Among the 39 miRNAs comprising the cell of origin classifier, 25 miRNAs are transcribed from miRNA clusters. This accounts for an enrichment in clusters compared with the whole dataset of probes analyzed in the microarray (64.1% vs. 45.5%, respectively). Furthermore, several miRNA paralog groups of clusters are represented in the cell of origin classifier. For example, miRNA paralog clusters miR-17/miR-92a-1, miR-363/miR-106a and miR-25/miR-106b are all highly expressed in centroblasts and follow similar profiles of expression across the different lymphocyte subpopulations (FIG. 2). Similarly, members of paralog clusters miR-16-1/miR-15a and miR-15b/miR-16-2, as well as the family of clusters of hsa-miR-181, are also up-regulated in centroblasts. In contrast, miRNAs from paralog clusters miR-24-2/miR-23a and miR-23b/miR-24-1, and miR-29a/miR-29-b-1 and miR-29c/miR-29-b-2 are up-regulated in memory B-cells compared to centroblasts.

miRNA Specifically Expressed in Cell Subpopulations Repress Expression of Genes with Roles in B-Cell Differentiation

The temporal variation in the levels of miRNA expression at distinct ontogeny stages of B lymphocytes raised a hypothesis that miRNAs may control expression of key proteins regulating peripheral B-cell maturation during immune response. Initially, miRNAs whose targets are known to change expression during B-cell differentiation were focused on. miR-15a and miR-16 are known to down-regulate the expression of BCL2. As expected, high expression of hsa-miR-15a and hsa-miR-16 in GC lymphocytes was detected, which are known not to express Bcl2 protein. Luciferase reporter assays using the 3′-UTR of the BCL2 gene confirmed repression of expression by hsa-miR-15a and hsa-miR-15b. These findings may explain downregulation of BCL2 expression in GC lymphocytes. Next, novel targets of miRNAs comprising the cell of origin classifier were explored by searching for putative binding sites in the 3′-UTR of genes with known roles in the GC reaction and differentiation to memory B-cells. Using three different prediction algorithms, it was found that putative binding sites for hsa-miR-223, highly expressed in naïve and memory B-cells but not in centroblasts, are harbored in the 3′-UTR of LMO2—a gene which is expressed at higher levels in GC lymphocytes. In addition, hsa-miR-125b, highly up-regulated in GC lymphocytes, has putative binding motifs in the 3′-UTRs of IRF4 and PRDM1—transcription factors necessary for post-GC maturation of the B lymphocytes.

To test regulation of these genes by the corresponding miRNAs, the precursor of hsa-miR-223 was transfected into VAL DLBCL cell line which expresses the LMO2 protein, and the precursor of hsa-miR-125b was transfected into RCK-8 DLBCL cell line which expresses the IRF4 and PRDM1 proteins. Western-blotting of whole cell lysates showed a decrease of native LMO2 in VAL cells transfected with hsa-miR-223 (FIG. 3A), when compared to control miRNA transfection with maximal decrease in LMO2 levels at 72h post transfection. hsa-miR-125b decreased expression levels of PRDM1 and IRF4 proteins at 48h after transfection (FIG. 3B). Examination of effects of hsa-miR-223 precursor on LMO2 mRNA revealed a decrease in expression at 24, 48 and 72h post transfection (FIG. 3C). hsa-miR-125b transfection had little effect on IRF4 mRNA levels, suggesting that the main regulation is at the protein translation level (FIG. 3D). Unexpectedly, hsa-miR-125b precursor-induced down-regulation of PRDM1 protein was associated with increased mRNA levels of PRDM1 transcript in RCK8 cells at 24, 48 and 72 h after transfection (FIG. 3D). Similarly, high levels of PRDM1 mRNA were previously observed in ABC-like DLBCL tumors not expressing PRDM1 protein due to nonsense PRDM1 mutations. These observations may suggest upregulation of PRDM1 mRNA in response to reduced expression of PRDM1 protein. Transfection efficacy in each experiment was confirmed by measurements of appropriate miRNAs by TaqMan MicroRNA Assays (FIGS. 3C and 3D, right panels).

It can be argued that hsa-miR-223 and hsa-miR-125b interact with other unknown targets, which in turn down-regulate levels of LMO2, IRF4 and PRDM1. To confirm direct effects, 3′-UTR sequences of LMO2, IRF4 and PRDM1, each containing putative binding sites to the corresponding miRNAs, were fused to a luciferase reporter gene. By co-transfecting the miRNA precursors with the corresponding constructs, it was demonstrated that all the tested miRNAs significantly repressed luciferase activity compared with a non-targeting control (FIG. 4). Taken together, these data support a direct effect of these miRNAs on their target transcripts and is in agreement with the results obtained for native proteins by Western-blot in DLBCL cell lines.

To further demonstrate the specificity of the interaction between the tested miRNAs and target mRNA sequences, a panel of reporter constructs containing the LMO2, IRF4 and PRDM1 3′-UTRs was generated with the corresponding miRNA putative binding sites mutated individually or in combination. The specific binding sites chosen for mutagenesis were selected based on analysis of accessibility of each putative miRNA binding site with the PITA algorithm. The following putative binding sites were selected for further studies: in the 3′-UTR of LMO2, two binding sites for hsa-miR-223 (positions 106 and 192 in the cloned 3′-UTR, ddG equal to -4.73 and -1.97, respectively), and two putative binding sites for hsa-miR-125b in each of the 3′-UTRs of PRDM1 (positions 39 and 1471, with associated ddG equal to −10.63 and −1.39) and IRF4 (positions 538 and 2160, with ddG of −6.36 and −0.65).

Mutagenesis of each putative binding site showed that all of them contributed to the regulation of these transcripts, though not to the same degree (FIG. 4). Cotransfection of the hsa-miR-223 precursor and the luciferase vector with the 3′-UTR of LMO2 into HeLa cells resulted in a reduction of luciferase activity to 51±4% when compared with a non-targeting control miRNA precursor. Mutagenesis of the seed of the most 5′ putative binding site for hsa-miR-223 induced a slight recovery of luciferase activity to 66±3% (not statistically significant). Mutation of the second putative site for this miRNA increased luciferase activity to 82±2%, and the combined mutation of both sites produced an almost complete restoration of luciferase activity (90±3%, not statistically different from the non targeting control) (FIG. 4A).

Co-transfection of the PRDM1 or IRF4 luciferase constructs with the precursor of hsa-miR-125b diminished luciferase activity to 51±2% for PRDM1 and 50±2% for IRF4 compared to the control. Mutagenesis of the most 5′ site in both 3′-UTRs induced recovery in luciferase activity to 67±2% for PRDM1 and 84±4% for IRF4, while mutagenesis of the most 3′ site induced no change compared to the wild type (52±3% for PRDM1 and 48±2% for IRF4). Nevertheless, combination mutagenesis of both sites restored luciferase activity to 79±5% for PRDM1 and 91±5% for IRF4, (FIG. 4B), suggesting a collaborative effect between the corresponding binding sites. In the case of the IRF4 double mutant there was almost complete abolishment of the regulation by hsa-miR-125b, while for PRDM1 double mutant there was a residual response to this miRNA, suggesting the possible existence of an additional functional binding site.

miRNA Expression in DLBCL Cell Lines

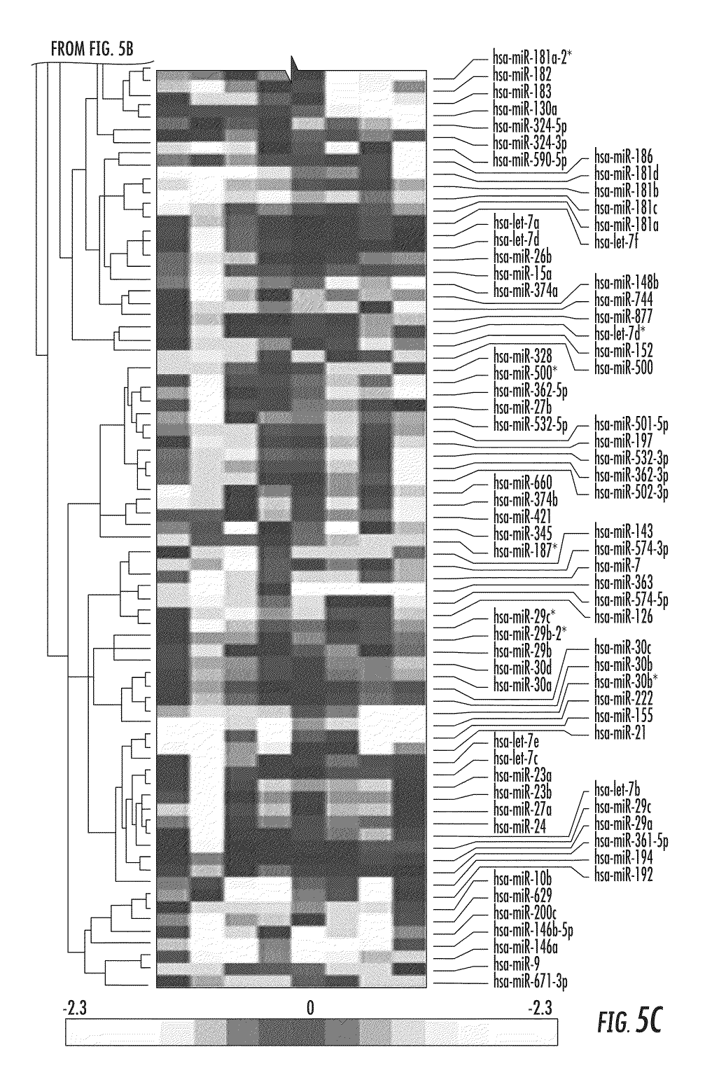

miRNA expression profiles in DLBCL cell lines representing the GCB-like and ABC-like lymphomas originating from GC and possibly post GC lymphocytes, respectively, were examined. miRNA samples from five ABC-like and three GCB-like DLBCL cell lines were labelled and hybridized to LC Sciences arrays representative of the 711 miRNAs known at the time of the experiment. To determine whether GCB-like and ABC-like cell lines could be distinguished based on their miRNA expression profiles, an unsupervised hierarchical clustering analysis was performed using the miRNA probes with data in at least 50% of samples (217 miRNAs). The clustering algorithm perfectly segregated GCB-like and ABC-like DLBCL cell lines based on global similarities in miRNA expression patterns (FIG. 5A). To identify a miRNA signature sufficient to separate GCB-like and ABC-like DLBCLs, a SAM analysis was performed using these 217 miRNA probes. This approach identified 9 miRNAs (hsa-miR-146b-5p, hsa-miR-146a, hsa-miR-21, hsa-miR-155, hsa-miR-500, hsa-miR-222, hsa-miR-363, hsa-miR-574-3p and hsa-miR-574-5p) differentially expressed between the DLBCL subtypes, at a FDR of 10%. (FIG. 5B). All of these 9 miRNAs were expressed at higher levels in the ABC-like DLBCL with differences up to 267 fold (hsa-miR-363). Interestingly, only 2 of these 9 miRNAs (hsa-miR-146b-5p and hsa-miR-146a) were included in the cell of origin classifier constructed to differentiate distinct normal B-cell subpopulations (FIG. 1B). This finding suggests that distinction between the GCB-like and ABC-like DLBCL subtypes is not solely based on distinct cellular origin but most probably also reflects different biology of these tumours.

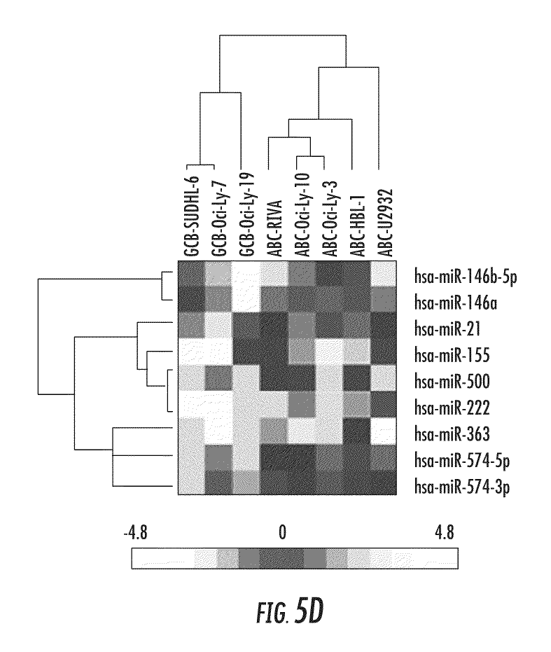

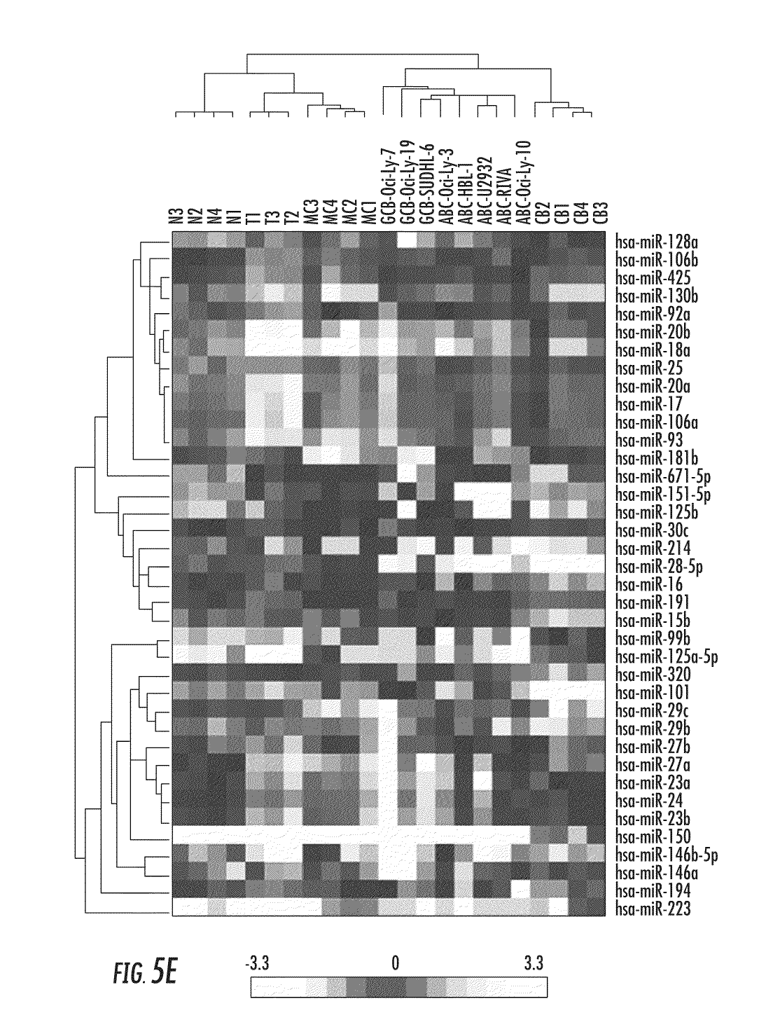

The similarities and differences in expression of miRNAs between normal lymphocyte subpopulations and GCB-like and ABC-like DLBCL cell lines were examined next. The 23 data sets corresponding to four samples each of the naïve, centroblasts and memory B-cells, three samples of T-cells and 8 samples of DLBCL cell lines were analyzed by unsupervised hierarchical clustering using the 39 miRNA cell of origin classifier. Again, the algorithm perfectly segregated individual normal B-cell subpopulations and T-cells. In addition, all the cell lines clustered together on a separate dendrogram branch with almost perfect segregation of ABC like and GCB-like cell lines (SUDHL-6 cell line was clustered in proximity to other GCB-like cell lines but on a dendrogram branch that also contained all the ABC-like cell lines). Interestingly, both the GCB-like and ABC-like cell lines clustered together with GC centroblasts on a major branch of the dendrogram, separated from memory B-cells, suggesting closer similarity in miRNA expression to GC lymphocytes (FIG. 5C). This finding suggests that based on miRNA profiling, GCB-like and ABC-like DLBCLs probably originate from distinct stages of GC lymphocytes. Of the 21 miRNA in the cell of origin classifier that are highly expressed in GC centroblasts, only 11 were also highly expressed in DLBCL cell lines, while the other 10 were expressed at significantly lower levels compared to normal cellular counterpart. Previous studies reported down-regulation of miRNAs in tumors compared to normal tissues (Lu et al., Nature. 435:834-838, 2005). To examine whether this phenomena is more general or is only restricted to GC specific miRNAs, a SAM analysis was performed using 143 miRNAs with data in at least 6 of 12 samples (4 GC specimens and 8 cell lines) to identify miRNA distinctively expressed between DLBCL cell lines and centroblasts, from which these tumors most probably originate. A total of 82 miRNAs showed differential expression with FDR of 10% (FIG. 6A). More than half of these miRNAs were up-regulated in cell lines. Among the 82 differentially expressed miRNAs, 28 belonged to the 39 miRNA comprising the cell of origin classifier and 2 (hsa-miR-21 and hsa-miR-574-3p) were included among the 9 miRNAs which expression was markedly different between the ABC and GCB-cell lines.

The next aim was to identify a classifier to differentiate centroblasts from DLBCL cells. To this end, a PAM analysis was performed that yielded a 5 miRNA classifier that differentiated normal centroblasts from DLBCL tumors without a single miss-classification error, as estimated by cross-validation (FIG. 6B).

hsa-miR-222 Expression Correlates with Outcome of DLBCL Patients

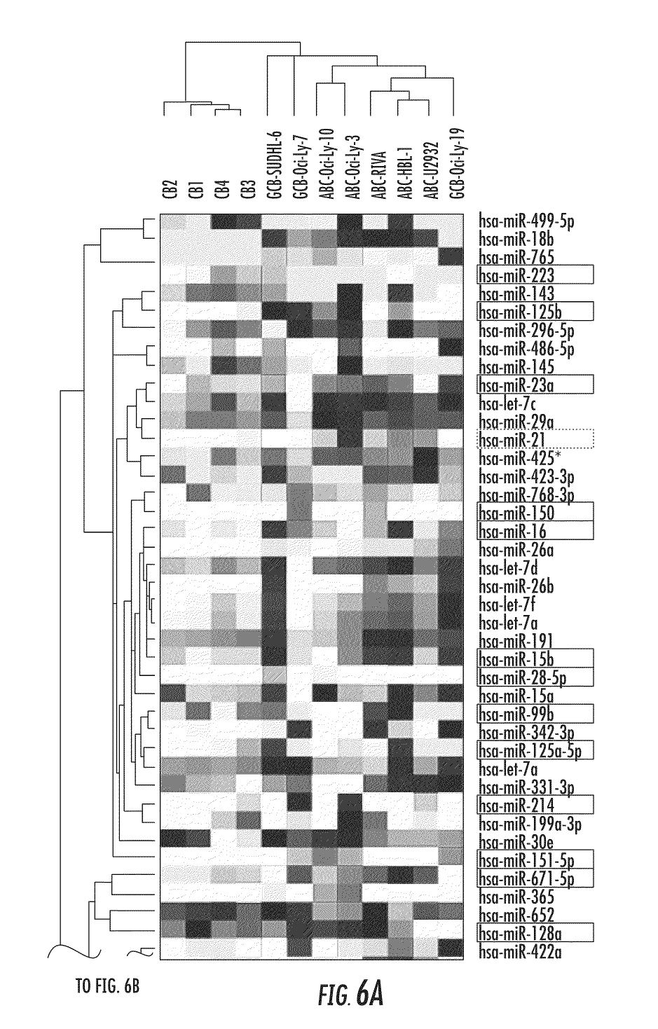

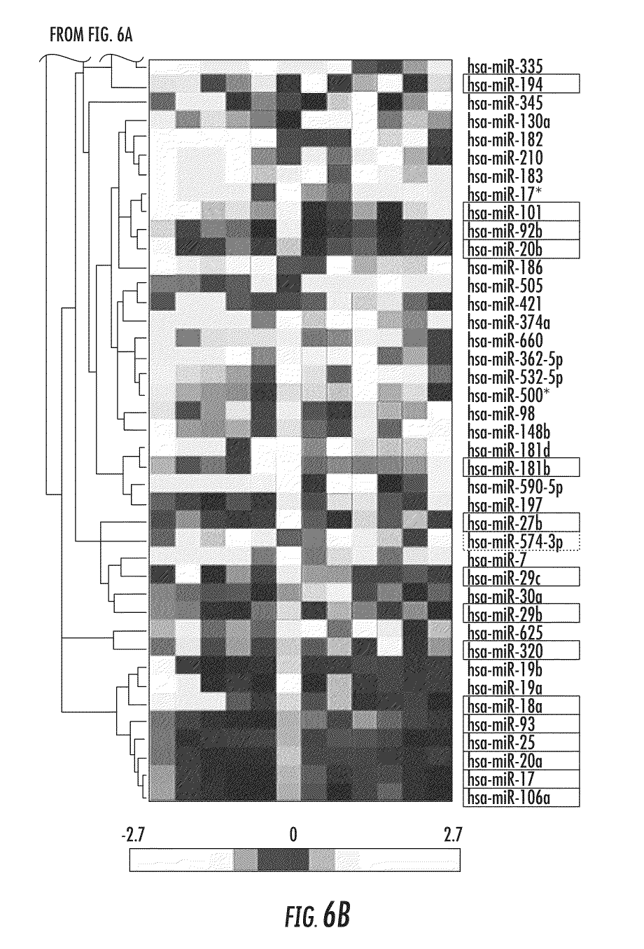

Previous studies demonstrated that patients with GCB-like and ABC-like DLBCL treated with cyclophosphamide, doxorubicin, vincristine and prednisone (CHOP) therapy demonstrate different survival (Rosenwald et al., N Engl J Med 346:1937-1947, 2002; Alizadeh et al., Nature 403:503-511, 2000). Moreover, expression of some, but not all of the genes characteristic of GCB and ABC gene signatures could be correlated with outcome of DLBCL patients (Lossos and Morgensztern J Clin Oncol. 24:995-1007, 2006). Therefore, expression of several of the miRNAs differentially expressed between GCB-like and ABC-like cell lines (FIG. 4B) was correlated to outcome of DLBCL patients. Expression of hsa-miR-222, hsa-miR-21, and hsa-miR-155 was examined in 106 specimens of DLBCL patients treated with current standard therapy: rituximab-CHOP(R-CHOP). The patients were divided into cases with low and high expression based on the median expression of each miRNA in the analyzed cohort. High expression of hsa-miR-222, characteristically expressed in ABC-like cell lines, was associated with inferior overall survival (OS) and progression free survival (PFS) (p=0.04 and p=0.03 respectively) (FIG. 7). hsa-miR-222 expression also predicted OS and PFS when considered as a continuous variable (P=0.0002 and 0.0005, respectively). Whether the prognostic significance of the hsa-miR-222 expression was independent of the international prognostic index (IPI) score was next examined. A multivariate Cox regression analysis that included IPI scores and hsa-miR-222 expression (as a continuous variable) with OS or PFS as the dependent variables was performed. Both the IPI and the hsa-miR-222 expression were independent predictors of OS (P=0.03 and 0.003, respectively) and PFS(P=0.003 and 0.007, respectively). No correlations between expression of hsa-miR-21or hsa-miR-155 and OS or PFS were observed.

miRNA profiles in B-cell subsets during peripheral B-cell differentiation as well as in diffuse large B-cell lymphoma (DLBCL) cells was examined. The results show temporal changes in the miRNA expression during B-cell differentiation with a highly unique miRNA profile in germinal center (GC) lymphocytes. Experimental evidence that these changes may be physiologically relevant was provided by demonstrating that GC-enriched hsa-miR-125b down-regulates the expression of IRF4 and PRDM1/BLIMP1 and memory B-cell enriched hsa-miR-223 down-regulates the expression of LMO2. It was further demonstrated that although an important component of the biology of a malignant cell is inherited from its non-transformed cellular progenitor—GC centroblasts—aberrant miRNA expression is acquired upon cell transformation. A 9 miRNAs signature was identified that could precisely differentiate the two major subtypes of DLBCL. Finally, expression of some of the miRNAs in this signature is correlated with clinical outcome of uniformly treated DLBCL patients.

### Example 2

### MicroRNA are Useful Biomarkers for Prediction of Response to Therapy and Survival of Patients with DLBCL

DLBCL is the most common subtype of non-Hodgkin's lymphoma. Despite its aggressive presentation, the current standard anthracycline-based immunochemotherapy regimen R-CHOP provides a complete remission rate of approximately 75%. Long-term disease-free survival is of only 50% displaying the wide heterogeneity of DLBCL. Traditional disease classification methods such as the International Prognostic Index, which is based on clinical characteristics, have provided some prognostic guidance. But the large variability of outcome within groups demonstrates that clinical or morphological features cannot account for all the heterogeneity of this disease underlying the need for novel prognostic biomarkers that may improve our upfront prognostication abilities.

Referring to the 6-gene Model, the 6 genes are LMO2, BCL6, BCL2, FN1, CCND2, and CCL3 (Lossos, NEJM, 2007). These 6 genes are listed below in Table 1

TABLE 1

| | | Number of | RR | P- | RR | P- |
|---|---|---|---|---|---|---|
| Authors | Treatment | Patients | (continuous) | value | (categorical) | value |
| Rosenwald et al. | CHOP | 240 | 1.14 | 0.015 | 1.62 | 0.005 |
| Shipp et al. | CHOP | 58 | 2.84 | 0.004 | 2.20 | 0.047 |
| Monti et al. | CHOP | 129 | 1.67 | 0.032 | 1.79 | 0.026 |
| Hummel et al. | CHOP | 81 | 2.45 | 0.013 | 2.10 | 0.020 |
| Lenz et al. | CHOP | 181 | 2.30 | 0.0002 | 1.96 | 0.0008 |
| | R-CHOP | 233 | 2.72 | 0.001 | 2.23 | 0.003 |
| Malumbres et al. | R-CHOP | 132 | 1.62 | 0.002 | 3.11 | 0.002 |

It was previously demonstrated that a prognostic score model based on the expression of 6 genes is able to predict clinical outcome independent of the IPI score. In 7 published cohorts the predictive power of the model has been confirmed with independence to the IPI score.

Micro-RNAs are short, non-protein coding RNAs of approximately 22 nucleotides in length. By targeting the 3′UTR region of messenger RNAs microRNAs modulate gene expression either by leading to translation repression or mRNA cleavage. By regulating gene expression microRNAs are postulated to play an important role in oncogenesis.

microRNA expression profile in lymphocyte differentiation was examined as described in Example 1 (see FIGS. 1b and 5b). It was previously demonstrated that microRNAs exhibit specific expression patterns in different stages of lymphocyte differentiation as is shown in the heat map on the left in which unsupervised hierarchical clustering subdivides centroblasts, T and B cells. We demonstrated that the DLBCL subtypes activated B-cell and germinal center B-cell type also exhibit different microRNA expression signatures. The heat map on the right demonstrates that a few miRs can accurately differentiate both GCB and ABC subtypes of DLBCL.

It was hypothesized that the expression of specific microRNAs can be used as a prognostic biomarker for prediction of outcome in DLBCL patients treated with the current standard regimen R-CHOP

### Patients and Methods

176 newly diagnosed DLBCL treated with R-CHOP
4 institutions (University of Miami, Stanford University, Universitat Autónoma de Barcelona, and British Columbia Cancer Agency)
RNA extracted from paraffin-embedded tissue (Chen et al. Diagn Mol Pathol, 2007)

RNA was extracted from paraffin-embedded archived tissues of newly diagnosed DLBCL lymphoma patients treated with R-CHOP whose clinical outcomes were known. RNA was extracted according to our previously published method. Patients were treated in one of the 4 institutions involved in the study while RNA extraction was performed at the University of Miami. In this study: n=176; median age 59 y (16-92) [84 patients 48%)>60 y]; 54 patients (30%)—ECOG PS≧2; 86 patients (49%)—Stage III-IV; and IPI [0-1—n=77, 2—n=50, 3—n=30, and 4-5—n=19]. The study group consisted of 176 patients with a median age 59 years with nearly half of patients older than 60 years. One third had an ECOG PS higher than 1 and half presented with advanced stage. Distribution according to the IPI demonstrated ⅔ of the patients with IPI of 0 to 2.

Referring to the microRNA and 6-gene Model, real-time PCR: miR-146a, miR-146b-5p, miR-222, miR-500, miR-574-3p, miR-363, miR-155, miR-21, miR-18a, miR-140-3p, miR-181a, LMO2, BCL-6, BCL-2, FN-1, CCND-2, and CCL3 (Example 1, and Lossos, NEJM, 2004). The expression of a total of 11 micro RNAs was analyzed by real-time PCR. These micro RNAs were selected based on their different expression between activated and germinal center B-cell types of DLBCL cell lines (point to 146a and b, 222, 500, 574-3p, 363,155, and 21) in the previous study or based on variable expression across DLBCL tumors as reported in the literature (point to miRs 140-3p, 181a, and 18a). The expression of the 6 genes which constitute the previously reported 6-gene model was also measured and the 6-gene survival score was calculated.

In an analysis of microRNA expression, Progression-free survival (PFS), Overall Survival (OS), and Statistical analysis [Continuous variable, Dichotomous model (2 groups−higher/lower than median value), Univariate analysis, and Multivariate analysis (microRNA+6−gene score+IPI score) was examined.

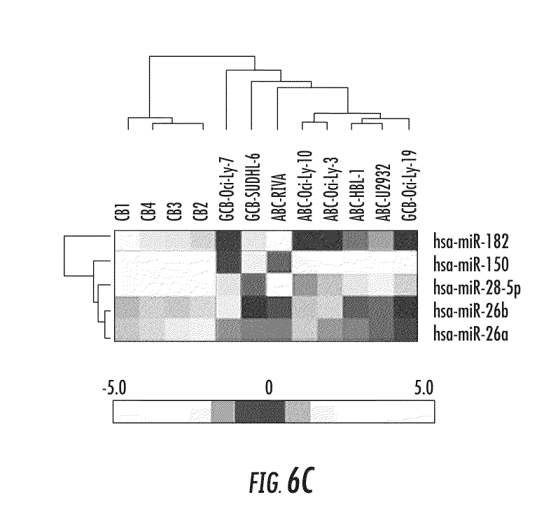

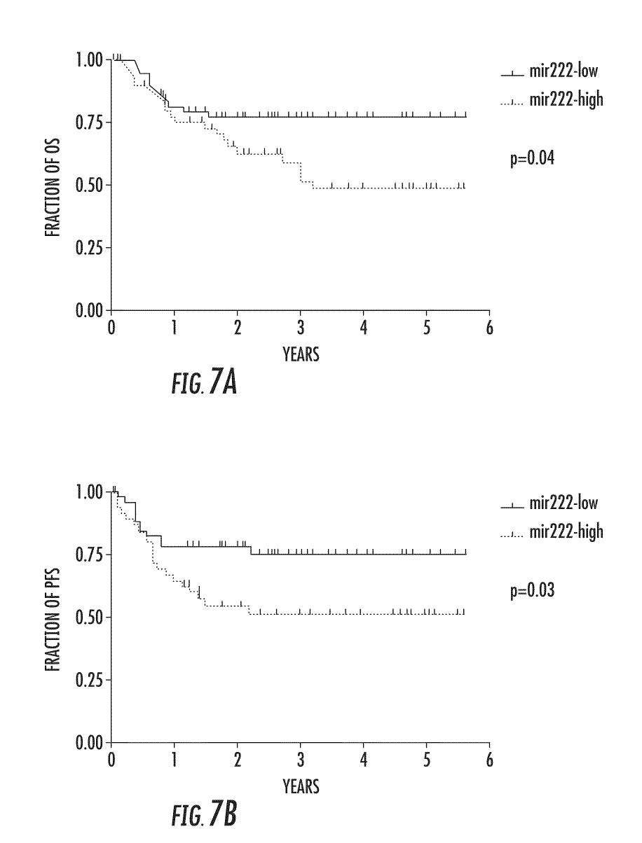

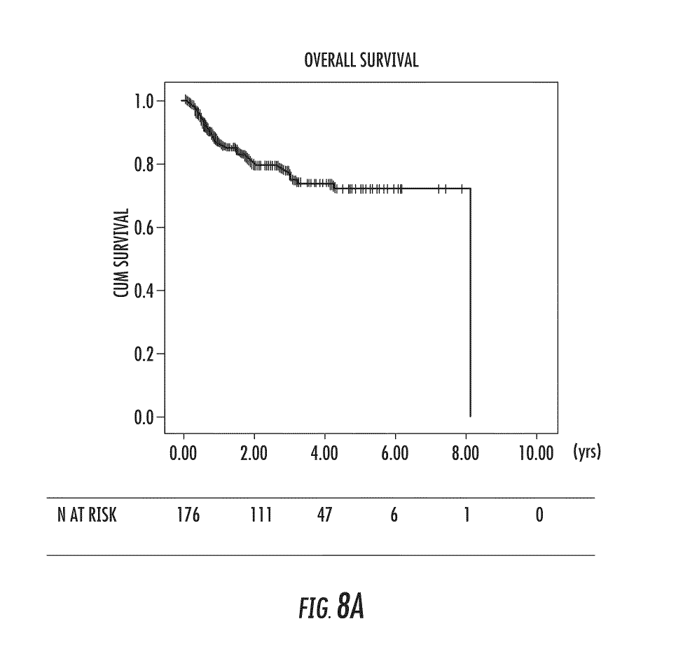

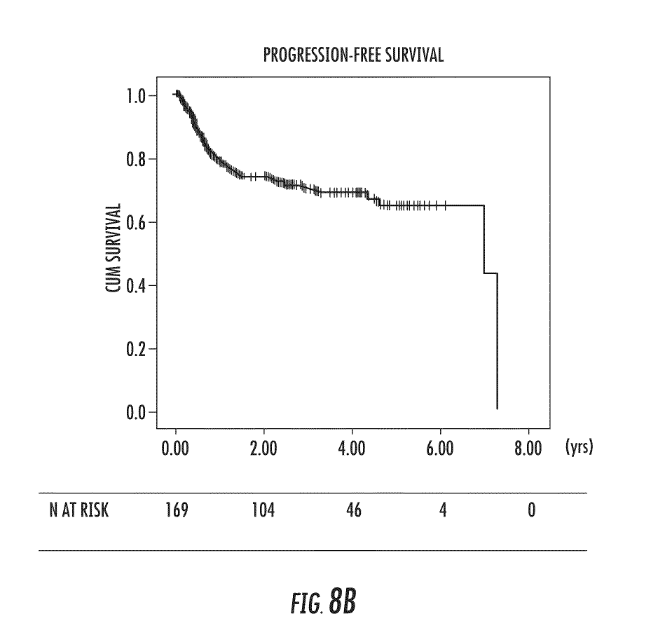

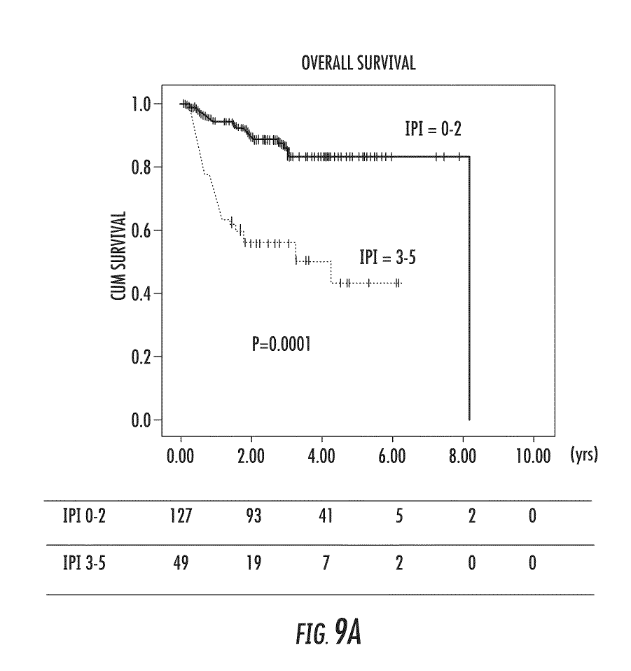

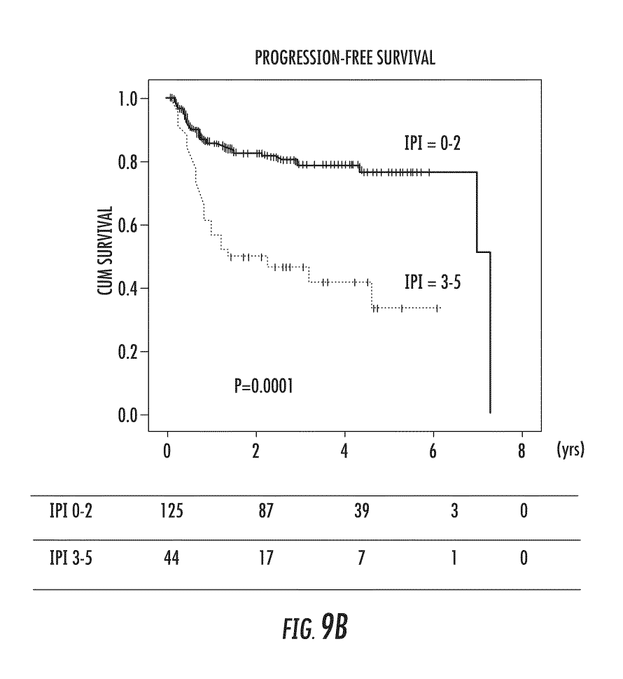

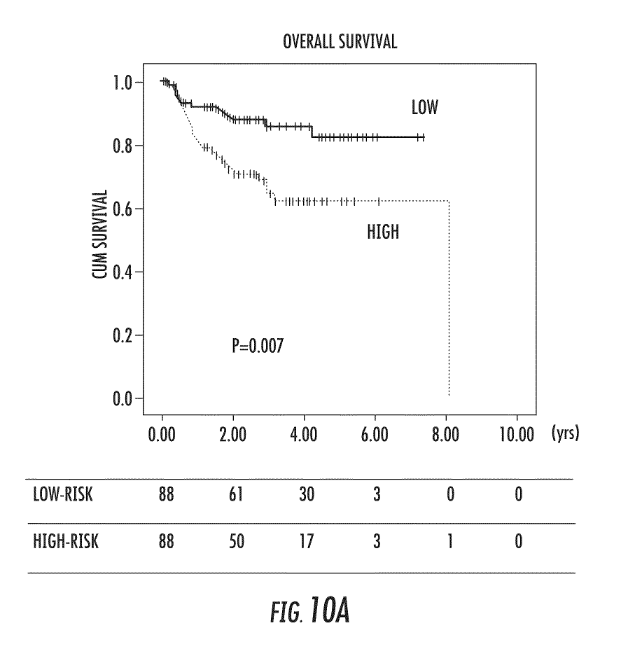

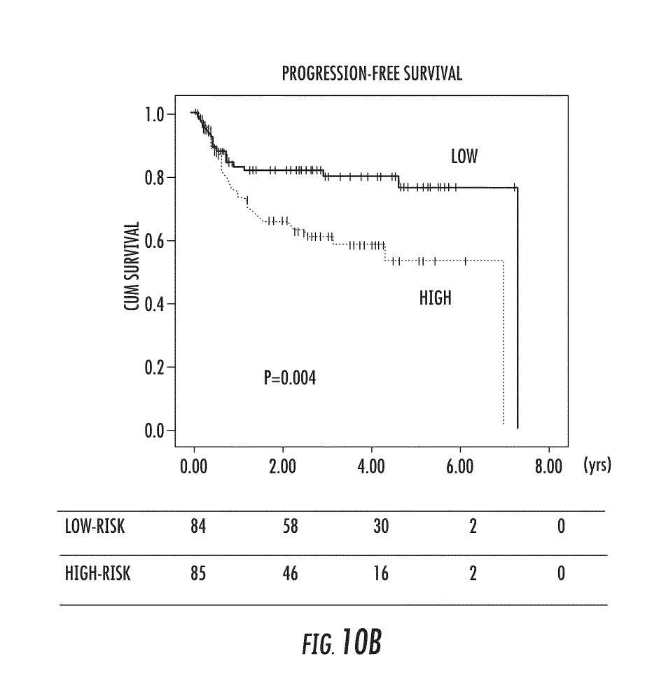

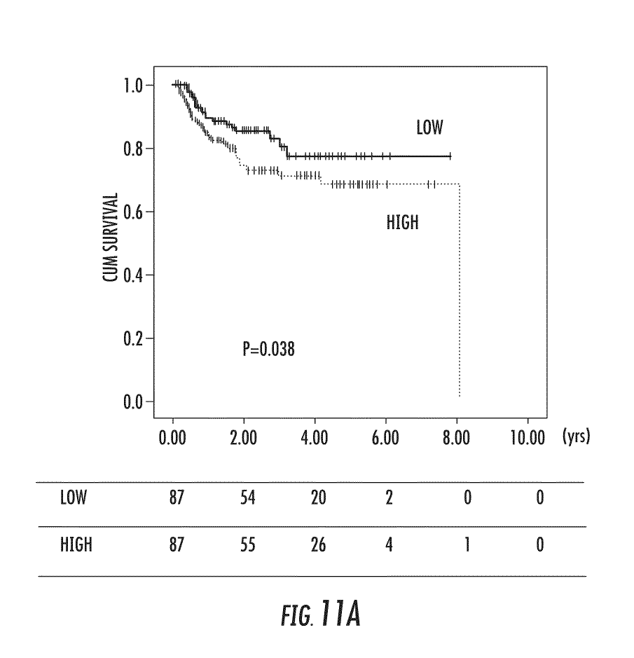

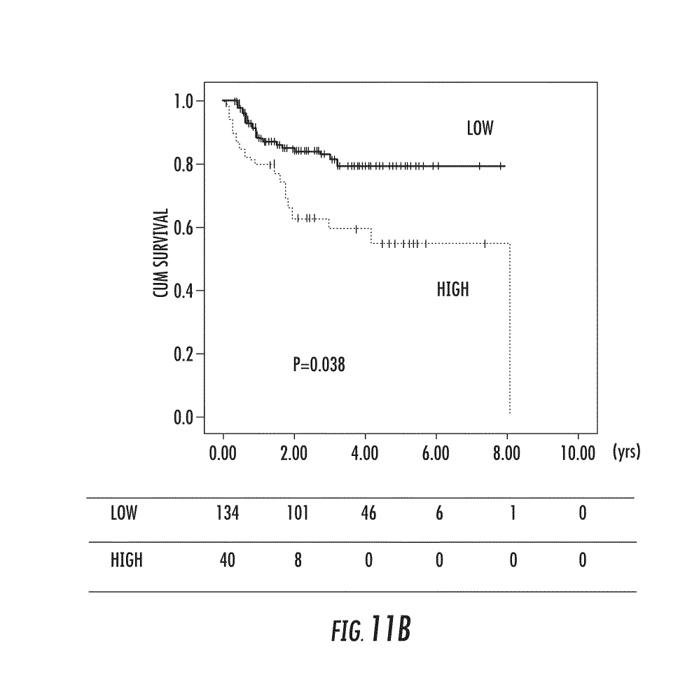

microRNA expression was then correlated to progression-free and overall survival. The predictive value of the expression was evaluated both as a continuous variable as well as in a dichotomous model in which patients were divided in 2 groups based on the microRNA median expression. Analysis was performed in univariate and multivariate methods. Multivariate analysis included the expression of the microRNAs as well as the IPI and 6-gene model scores. The results are shown in FIGS. 8-13. Referring to FIG. 8, during a median follow-up of 2.6 years a total of 54 patients had documented progression of disease. A total of 41 deaths were documented. Referring to FIG. 9, IPI score was predictive of both OS and PFS in the cohort of patients. Referring to FIG. 10, the 6-gene model was also predictive of both OS and PFS in the cohort of patients thus confirming its robustness in an additional cohort of patients. The following 8 microRNAs were not associated with outcome: miR-146a, miR-146b-5p, miR-500, miR-574-3p, miR-363, miR-155, miR-21, and miR-140-3p. Referring to FIG. 11, microRNA-18a expression was analyzed, and expression was associated with OS as continuous variable, and high expression correlated with a worse outcome. 3 of the 11 assessed were significantly associated with clinical outcome. Expression of miR-18a when evaluated as a continuous variable significantly correlated with overall survival. Patients with higher expression demonstrated inferior outcomes. Since the variable is continuous, two different Survival curves using two representative categorical cut-offs are demonstrated. Expression of miR-18a did not correlate with PFS. Referring to FIG. 12, microRNA-181a expression was analyzed. Expression was associated with PFS as continuous variable, and high expression correlated with a better outcome. Expression of miR-181a when evaluated as a continuous variable correlated with PFS but not with OS. Higher expressions were associated with longer PFS. Since the variable is continuous, two different Survival curves were demonstrated using two representative categorical cut-offs. Referring to FIG. 13, miR-222 expression was examined and associated with PFS as categorical variable. Low expression correlated with a better outcome. miR-222, when evaluated as a categorical variable subdividing the groups based on median expression was associated with PFS. Lower expression was associated with longer PFS as demonstrated on the graphic.

Referring to Table 2, in a multivariable model analysis including IPI, 6-gene model and miRs 18a, 181a, and 222, all the variables, except expression of mir222 were independent predictors of OS

TABLE 2
6 gene + IPI + miRs

| | z score | p score |
|---|---|---|
| IPI score | 4.12 | <0.005 |
| 6-gene | 2.11 | 0.035 |
| miR-18a | 2.53 | 0.011 |
| miR-181a | −2.88 | 0.004 |
| miR-222 | 1.06 | NS |

Referring to Table 3, in a multivariable model analysis including IPI, 6-gene model and miRs 18a, 181a, and 222, all the variables, except expression of mir18a were independent predictors of PFS. These findings suggest that expression of specific mirs may contribute to the predictive power of IPI and 6-gene model.

TABLE 3
Multivariate Analysis for PFS: 6-gene + IPI score + miRs

| | z score | p score |
|---|---|---|
| IPI score | 3.52 | <0.005 |
| 6-gene | 1.95 | 0.05 |
| miR-18a | 0.81 | NS |
| miR-181a | −2.79 | 0.005 |
| miR-222 | 2.65 | 0.008 |

In conclusion, expression of miR-18a, miR-181a, and miR-222 is associated with outcome of patients with DLBCL and is independent of IPI and 6-gene model. In RCHOP treated patients with DLBCL, expression of miRNAs miR-18a, miR-181a and miR-222 is associated with response to therapy and outcome, independent of both the IPI and the mortality-prediction score calculated from the 6-gene model. While the expression of miR-181a and miR-222 may reflect the cell of origin of DLBCL tumors as previously described, the expression of miR-18a does not.

### Example 3

### Expression Studies of miR-181a

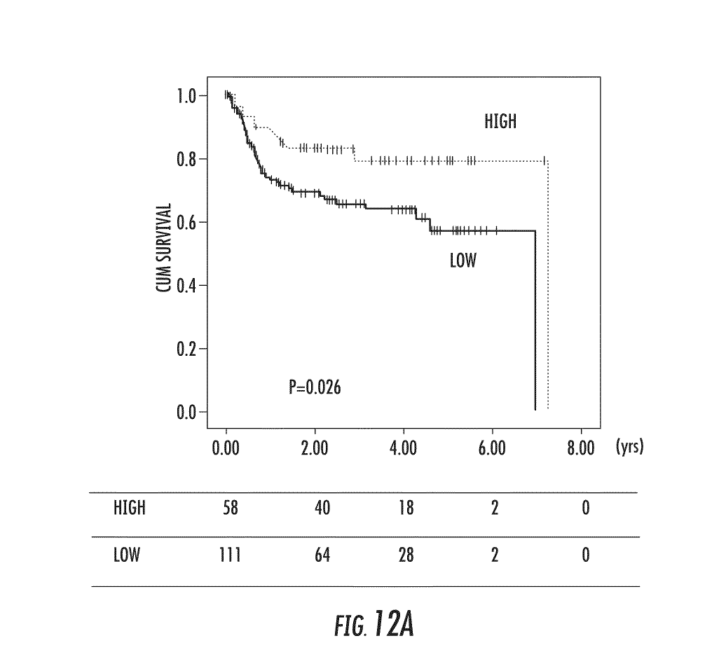

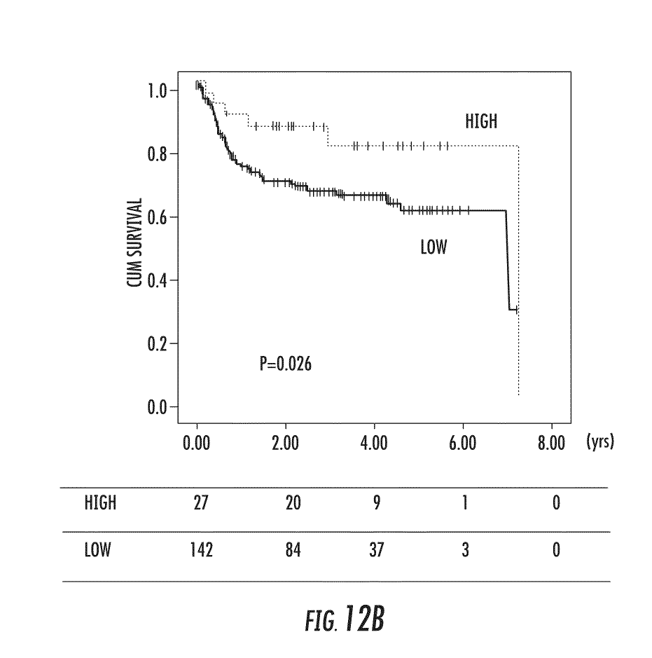

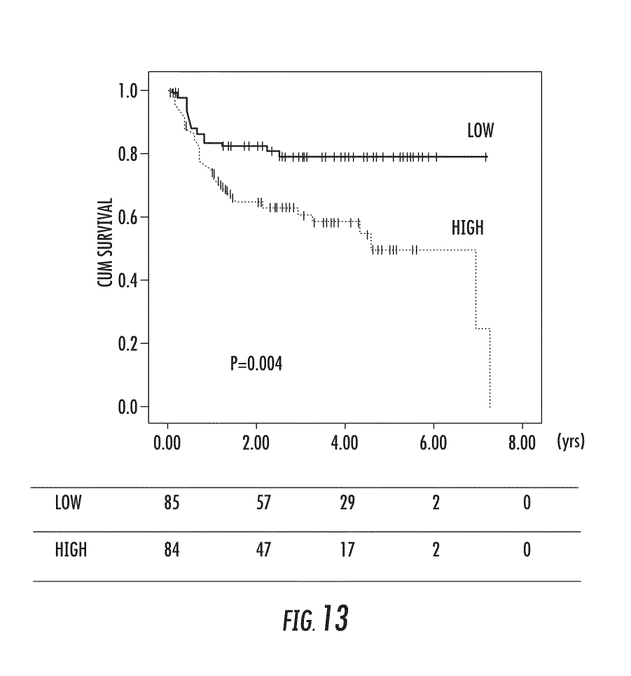

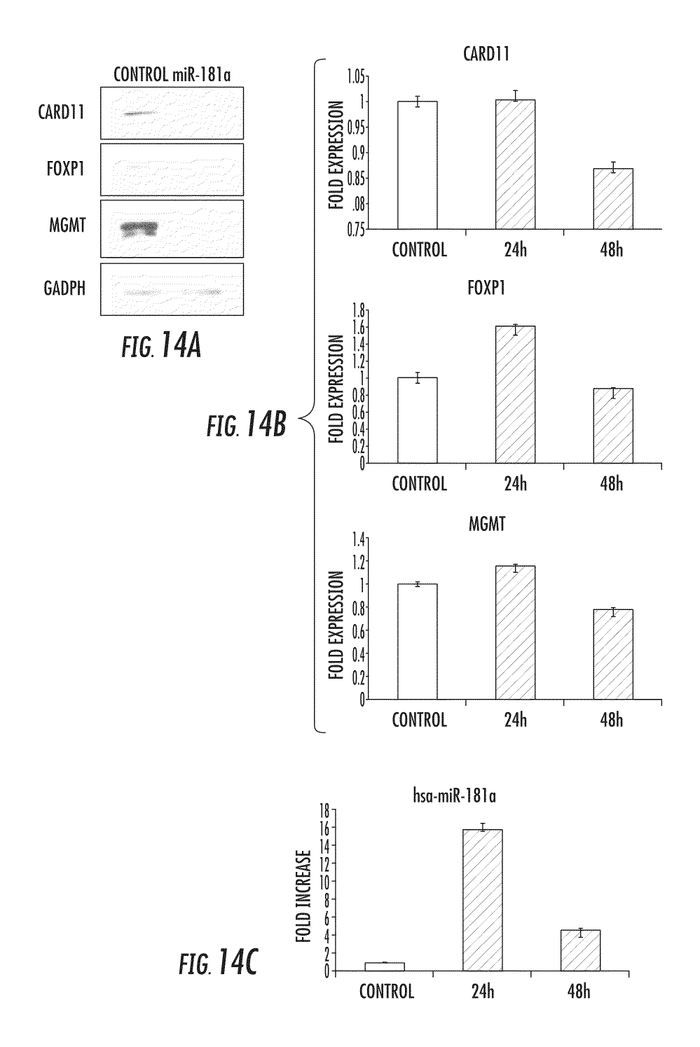

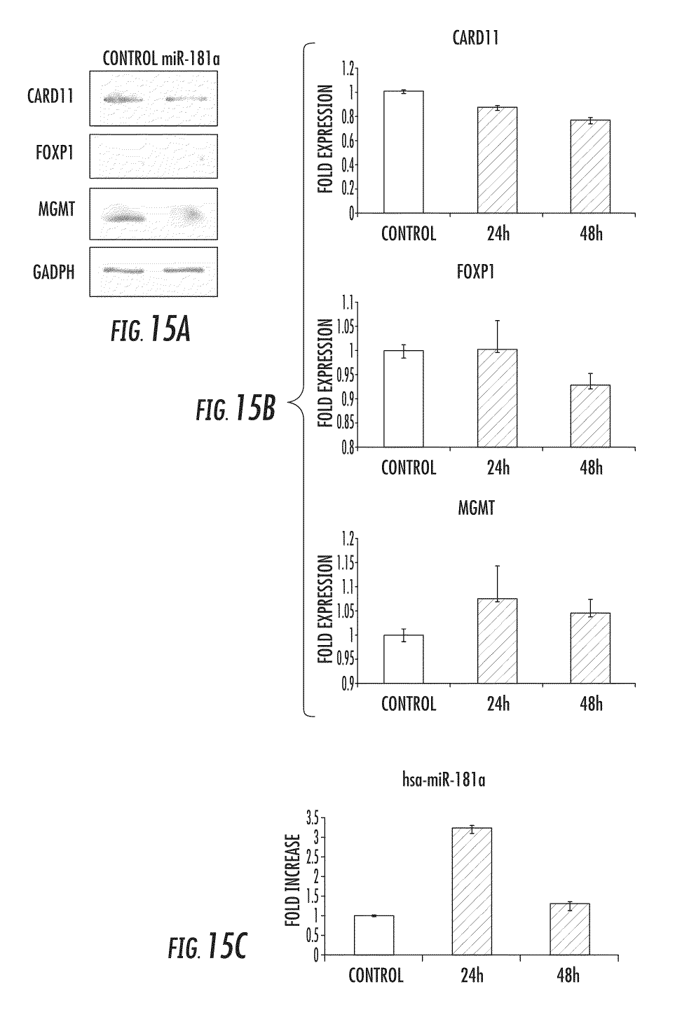

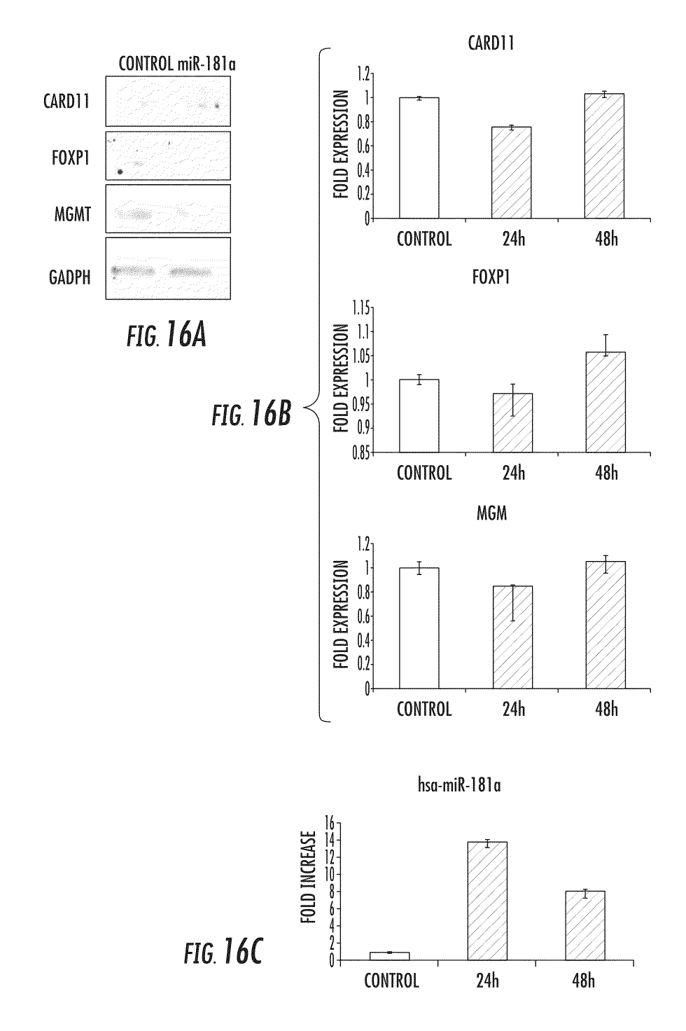

The effects of has-miR-181a expression on protein and mRNA levels of genes with roles in DLBCL-patients' survival were examined. FIGS. 14-16 show the effect of has-miR-181a expression on native CARD11, FOXP1, and MGMT protein levels in lymphoma cell lines (VAL cells (FIG. 14A), Jukart cells (FIG. 15A), Raji cells (FIG. 16A)) at 24 hours after transfection, assessed by Western blot analyses. GADPH levels were used as loading control in all cases. Data were confirmed in triplicate experiments. FIGS. 14B, 15B, and 16B show the effects of the expression of has-miR-181a on the mRNA levels of CARD11, FOXP1, and MGMT measured by real-time PCR using TaqMan Gene Expression Assays (Applied Biosystems) at 24 and 48 hours after transfection. Values of triplicate wells are represented as fold expression with respect to the non-targeting control transfection. Expression of has-miR-181a was confirmed by TaqMan MicroRNA Assays (FIGS. 14C, 15C and 16C), expressed as fold-increase regarding the control transfection. Error bars correspond to standard error of the mean in all graphs.

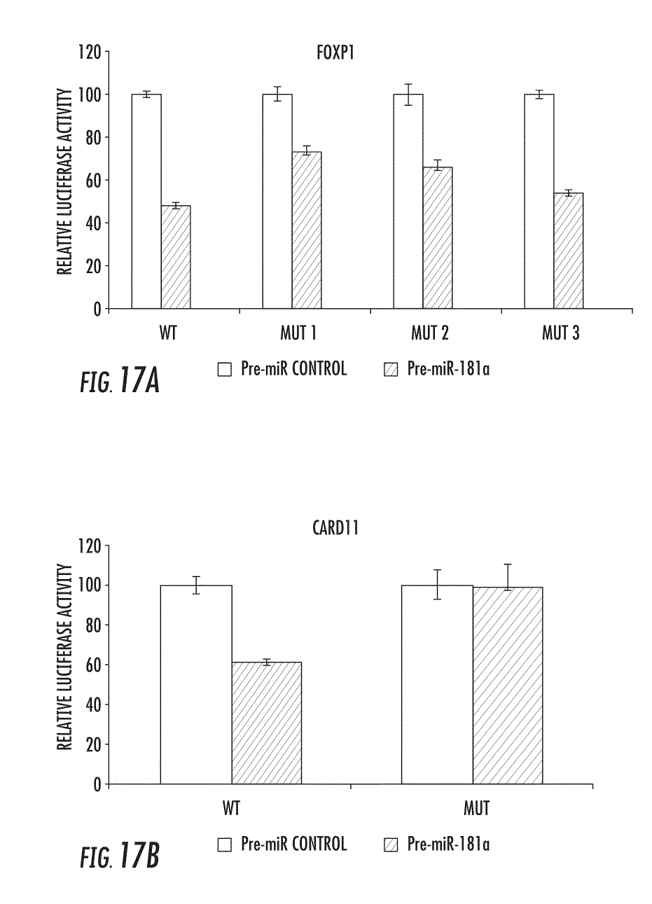

Referring to FIG. 17, hsa-miR-181a regulates FOXP1 and CARD11 by binding to cis-regulatory elements in the 3′-UTRs of FOXP1 and CARD11. FIG. 17A shows dual luciferase activity of reporter plasmids with the wild-type or mutated 3′-UTR of FOXP1 fused to the luciferese gene upon has-miR-181a precursor co-transfection in HeLa cells. Mutations of the putative binding sites of FOXP1 are numbered MUT1-MUT3, with MUT1 representing the most 5′ site, and MUT3 the most 3′ site. The same experiment was performed for CARD11 (FIG. 17B). Results are reported as ratios of luciferase to Renilla activity normalized to the value of each control, which is noted as 100%. Error bars correspond to the SEM.

### Other Embodiments

Any improvement may be made in part or all of the assays, kits, and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. An assay for predicting survival outcome in a subject having diffuse large B-cell lymphoma (DLBCL), the assay comprising:
(a) analyzing expression of miR-18a, miR-181a and miR-222 in a biological sample from the subject; and
(b) correlating at least one of: decreased expression of miR-18a in the biological sample compared to a control sample, increased expression of miR-181a in the biological sample compared to a control sample, and decreased expression of miR-222 in the biological sample compared to a control sample with increased survival.

2. The assay of claim 1, wherein step (b) comprises correlating decreased expression of miR-18a in the biological sample compared to a control sample, increased expression of miR-181a in the biological sample compared to a control sample, and decreased expression of miR-222 in the biological sample compared to a control sample with increased survival.

3. The assay of claim 1, wherein the biological sample is a frozen specimen or a paraffin embedded specimen.

4. The assay of claim 1, wherein the outcome is predicted within 24 hours of the subject being diagnosed with DLBCL.

5. The assay of claim 1, wherein expression of miR-18a, miR-181a and miR-222 is performed using real-time PCR.

4. The assay of claim 1, further comprising analyzing expression of genes LMO2, BCL-6, BCL-2, FN-1, CCND-2, and CCL3 and calculating a mortality prediction score.

5. The assay of claim 4, further comprising determining an International Prognostic Index (IPI) score.

6. The assay of claim 5, wherein the assay comprises a multivariate analysis.

7. The assay of claim 1, wherein the subject is a human.

8. A method for measuring a response to DLBCL therapy in a subject having DLBCL, the method comprising:
(a) analyzing expression of miR-18a, miR-181a and miR-222 in a biological sample obtained from the subject at a first time point;
(b) analyzing expression of miR-18a, miR-181a and miR-222 in a biological sample obtained from the subject at a second time point,
wherein the subject receives the DLBCL therapy after the first time point and before the second time point;
(c) comparing the expression of miR-18a, miR-181a and miR-222 in the biological sample obtained from the subject at a first time point to the expression of miR-18a, miR-181a and miR-222 in the biological sample obtained from the subject at a second time point; and
(d) correlating at least one of: decreased expression of miR-18a in the biological sample obtained from the subject at a second time point compared to the sample obtained from the subject at a first time point, increased expression of miR-181a in the biological sample obtained from the subject at a second time point compared to the sample obtained from the subject at a first time point, and decreased expression of miR-222 in the biological sample obtained from the subject at a second time point compared to the sample obtained from the subject at a first time point with a therapeutic response to the therapy.

9. The method of claim 8, wherein the therapy comprises R-CHOP.

10. The method of claim 8, wherein the subject is a human.

11. The method of claim 8, wherein the sample obtained from the subject at the first and second time points is a frozen specimen or a paraffin embedded specimen.

12. A kit for predicting survival outcome in a subject having DLBCL, the kit comprising:
(a) at least one reagent for analyzing expression of miR-18a, miR-181a and miR-222 in a biological sample from the subject;
(b) at least one control; and
(c) instructions for use.
